SEQUENCE LISTING (2 sequences)

SEQ ID NO: 1 - DNA, 81 bp, Human
ctccatctcctccactgttcccccacactgtgctgtgacaatgagatgagacagagggtcaggacaacatcaaggggtgta

SEQ ID NO: 2 - DNA, 81 bp, Human
aaagacataaacctccctgtgactccattttggtaactgtatccaaaacacaggatccctgctgttctttgtttcctttta

(19) United States
(12) Patent Application Publication
(10) Pub. No.: US 2006/0014189 A1
(43) Pub. Date: Jan. 19, 2006

(54) Controls for determining reaction performance in polynucleotide sequence detection assays

(75) Inventors: H. Michael Wenz, Redwood City, CA (US); Joseph P. Day, Sonora, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/171,107

(22) Filed: Jun. 29, 2005

Related U.S. Application Data
(60) Provisional application No. 60/584,873, filed on Jun. 30, 2004.

(51) Int. Cl. C12Q 1/68

(52) U.S. Cl. 435/6

(57) ABSTRACT

The present teachings relate to methods, compositions, and kits for detecting one or more target polynucleotide sequences in a sample. In some embodiments of the present teachings, probes are hybridized to complementary target polynucleotides and are ligated together to form a ligation product. Some embodiments of the present teachings comprise positive assay control probes that provide information regarding the occurrence of specific ligation in a complex ligation assay mixture. Some embodiments of the present teachings provide for negative assay control probes that provide information regarding the occurrence of non-specific ligation in a complex ligation assay mixture. Some embodiments of the present teachings provide for the generation of two distinct signals from a monomorphic target polynucleotide sequence.

### CROSS REFERENCE TO RELATED APPLICATIONS

This application claims priority benefit under 35 U.S.C. § 119(e) from U.S. Provisional Patent Application No. 60/584,873, filed Jun. 30, 2004, which is incorporated herein by reference.

### FIELD

The present teachings generally relate to methods, kits, and compositions for detecting one or more target polynucleotide sequences in a sample. More specifically, the methods, kits, and compositions employ positive controls and negative controls for determining reaction performance in polynucleotide sequence detection assays.

### BACKGROUND

The detection of the presence or absence of (or quantity of) one or more target polynucleotides in a sample or samples containing one or more target sequences is commonly practiced. For example, the detection of cancer and many infectious diseases, such as AIDS and hepatitis, routinely includes screening biological samples for the presence or absence of diagnostic nucleic acid sequences. Also, detecting the presence or absence of nucleic acid sequences is often used in forensic science, paternity testing, genetic counseling, and organ transplantation.

An organism's genetic makeup is determined by the genes contained within the genome of that organism. Genes are composed of long strands or deoxyribonucleic acid (DNA) polymers that encode the information needed to make proteins. Properties, capabilities, and traits of an organism often are related to the types and amounts of proteins that are, or are not, being produced by that organism.

A protein can be produced from a gene as follows. First, the information that represents the DNA of the gene that encodes a protein, for example, protein “X”, is converted into ribonucleic acid (RNA) by a process known as “transcription.” During transcription, a single-stranded complementary RNA copy of the gene is made. Next, this RNA copy, referred to as protein X messenger RNA (mRNA), is used by the cell's biochemical machinery to make protein X, a process referred to as “translation.” Basically, the cell's protein manufacturing machinery binds to the mRNA, “reads” the RNA code, and “translates” it into the amino acid sequence of protein X. In summary, DNA is transcribed to make mRNA, which is translated to make proteins.

The amount of protein X that is produced by a cell often is largely dependent on the amount of protein X mRNA that is present within the cell. The amount of protein X mRNA within a cell is due, at least in part, to the degree to which gene X is expressed. Whether a particular gene or gene variant is present, and if so, with how many copies, can have significant impact on an organism. Whether a particular gene or gene variant is expressed, and if so, to what level, can have a significant impact on the organism.

### SUMMARY

In some embodiments, the present teachings provide a method for producing more than one signal from a monomorphic target polynucleotide sequence comprising, providing the monomorphic target polynucleotide sequence and a positive control first probe one and a positive control first probe two, wherein the positive control first probe one comprises a target specific portion complementary to the monomorphic polynucleotide sequence, and an identifying portion, wherein the positive control first probe two comprises a target specific portion complementary to the monomorphic target polynucleotide sequence, and an identifying portion, wherein the identifying portion of the positive control first probe one differs from the identifying portion of the positive control first probe two; hybridizing the positive control first probe one and the positive control first probe two to the monomorphic target polynucleotide sequence; separating the hybridized probes from the unhybridized probes; detecting the identifying portions of the positive control first probe one and the positive control first probe two that hybridized to the monomorphic target polynucleotide sequence, thereby producing more than one signal from the monomorphic target polynucleotide sequence.

In some embodiments, the present teachings provide a method for assessing ligation specificity in a ligation assay comprising; providing a monomorphic target polynucleotide sequence and a control probe set, wherein the control probe set comprises a positive control first probe and a negative control first probe, and a second probe, wherein the positive control first probe comprises a target specific portion, wherein the target specific portion comprises a discriminating region, and an identifying portion, wherein the negative control first probe comprises a target specific portion, wherein the target specific portion comprises a discriminating region, and an identifying portion, wherein the identifying portion of the positive control first probe and the negative control first probe are different, wherein the second probe of the control probe set comprises a target specific portion; hybridizing the first positive control probe, the first negative control probe, and the second probe to the monomorphic target polynucleotide sequence, wherein the positive control probe and the negative control probes can hybridize adjacently to the second probe on the monomorphic target polynucleotide sequence, wherein the discriminating region of the positive control probe allows complete complementarity with the monomorphic target polynucleotide, wherein the discriminating region of the negative control probes prevents complete complementarity with the monomorphic target polynucleotide; ligating the positive control first probe to the second probe, ligating the negative control first probe to the second probe, thereby generating a specific ligation product and a non-specific ligation product comprising different identifying portions; separating the non-specific ligation products and the specific ligation products from the unhybridized control probes and unligated control probes; detecting the specific and non-specific ligation products based on their distinct identifying portions; comparing the amount of specific ligation products to non-specific ligation products; thereby assessing ligation in a ligation assay.

In some embodiments, the methods of the present teachings further comprise a polymorphic polynucleotide target sequence and an experimental probe set, wherein the experimental probe set comprises an experimental first probe one, an experimental first probe two, and an experimental second probe, wherein the experimental first probe one comprises an identifying portion and a target specific portion complementary to the polymorphic polynucleotide target sequence, wherein the target specific portion comprises a discriminating region, wherein the experimental first probe two comprises an identifying portion and a target specific portion complementary to the polymorphic polynucleotide target sequence, wherein the target specific portion comprises a discriminating region, wherein the identifying portion of the experimental first probe one differs from the identifying portion of the experimental first probe two, wherein the discriminating region of the experimental first probe one differs from the discriminating region of the experimental first probe two, wherein the second experimental probe comprises a target specific portion complementary to the polymorphic polynucleotide target sequence, wherein the discriminating region of the experimental first probes can hybridize with different nucleotides corresponding to different alleles of a single nucleotide polymorphism, wherein the experimental first probes are hybridized adjacent to the experimental second probe on the polymorphic target polynucleotide sequence; ligating the experimental first probe one to the contiguously hybridized experimental second probe, ligating the experimental first probe two to the contiguously hybridized second probe, thereby generating specific ligation products comprising different identifying portions corresponding to two alleles of a single nucleotide polymorphism; separating the specific ligation products and the non-specific ligation products from the unhybridized experimental first probes and experimental second probes, and unligated experimental first probes and experimental second probes; detecting the specific and non-specific ligation products based on their distinct identifying portions; comparing the amount of specific ligation products to non-specific ligation products resulting from the control probe set; comparing the ligation products resulting from the control probe set to the ligation products resulting from the experimental probe set, thereby assessing ligation in a ligation assay.

In some embodiments, the present teachings provide a method for assessing ligation comprising; providing a first reaction comprising a monomorphic target polynucleotide sequence and a positive control probe set, wherein the positive control probe set comprises a positive control first probe one, a positive control first probe two, and a positive control second probe, wherein the positive control first probe one comprises an identifying portion and a target specific portion complementary to the monomorphic polynucleotide sequence, wherein the target specific portion comprises a discriminating region complementary to the corresponding nucleotide on the monomorphic polynucleotide sequence, wherein the positive control first probe two comprises an identifying portion and a target specific portion complementary to the monomorphic polynucleotide sequence, wherein the target specific portion comprises a discriminating region complementary to the corresponding nucleotide on the monomorphic polynucleotide sequence, wherein the identifying portion of the positive control first probe one differs from the identifying portion of the positive control first probe two, wherein the positive control second probe comprises a target specific portion; hybridizing the monomorphic target polynucleotide sequence to the positive control first probe one, the positive control first probe two, and the positive control second probe, wherein the positive control first probes hybridize adjacent to the second probe; ligating the positive control first probe one to the positive control second probe, ligating the positive control first probe two to the positive control second probe, thereby forming a positive control first probe one ligation product and a positive control first probe two ligation product; detecting the positive control first probe one ligation product and the positive control first probe two ligation product based on their distinct identifying portions, thereby assessing ligation.

Some embodiments of the present teachings further comprise a second reaction, wherein the second reaction comprises a monomorphic target polynucleotide sequence and a negative control probe set, wherein the negative control probe set comprises a negative control first probe one, a negative control first probe two, and a negative control second probe, wherein the negative control first probe one comprises an identifying portion and a target specific portion complementary to the monomorphic polynucleotide sequence, wherein the target specific portion comprises a discriminating region that is not complementary to the corresponding nucleotide of the monomorphic target polynucleotide, wherein the negative control first probe two comprises an identifying portion and a target specific portion complementary to the monomorphic polynucleotide sequence, wherein the target specific portion further comprises a discriminating region that is not complementary to the corresponding nucleotide of the monomorphic target polynucleotide, wherein the identifying portion of the negative control first probe one differs from the identifying portion of the negative control first probe two, wherein the negative control second probe comprises a target specific portion; hybridizing the monomorphic target polynucleotide sequence to the negative control first probe one, the negative control first probe two, and the negative control second probe, wherein the negative control first probes hybridize adjacent to the second probe; ligating the negative control first probe one to the negative control second probe, ligating the negative control first probe two to the negative control second probe, thereby forming a negative control first probe one ligation product and a negative control first probe two ligation product; detecting the negative control first probe one ligation product and the negative control first probe two ligation product based on their distinct identifying portions; comparing the detected non-specific ligation product in the first reaction to the detected specific ligation products in the second reaction, thereby assessing non-specific ligation in a ligation assay.

In some embodiments, the ligation products are amplified by a PCR.

In some embodiments, the PCR comprises an affinity moiety-labeled primer.

In some embodiments, the identifying portions of the probes are incorporated in the resulting PCR amplicons, the method further comprising; immobilizing affinity moiety-labeled amplicon strands with an affinity moiety binding partner; removing reaction components lacking the affinity moiety; hybridizing a plurality of mobility probes to the immobilized affinity moiety-labeled amplicon strands, wherein the mobility probes further comprise a region of complementary with the identifying portion or identifying portion complement of the affinity moiety-labeled amplicon strands; removing unhybridized mobility probes; eluting hybridized mobility probes; and, analyzing the eluted mobility probes using a mobility dependent analysis technique, whereby the distinct mobility of the mobility probe determines the identity of the identifying portion, and hence an assessment of ligation.

In some embodiments the mobility probes further comprise distinguishable labels, wherein said distinguishable labels further comprise at least one florophore, wherein the florophore is at least one of 6FAM, dR6G, BigDye-Tamra, BigDye-Rox, and combinations thereof.

In some embodiments, the mobility dependent analysis technique is capillary electrophoresis.

In some embodiments, the affinity moiety is biotin.

In some embodiments, the affinity moiety-binding partner is streptavidin.

In some embodiments, a universal forward primer portion is incorporated into the first probes, wherein a universal reverse primer portion is incorporated into the second probes, wherein the PCR amplification comprises a set of universal primers that hybridize to their corresponding primer portions.

In some embodiments, the present teachings provide a method for determining ligation specificity in a ligation assay comprising; comparing the amount of a specific positive control ligation product to a non-specific negative control ligation product, wherein the specific positive control ligation product results from ligating a first positive control probe to a second probe while hybridized on a monomorphic target polynucleotide, wherein the non-specific negative control ligation product results from ligating a first negative control probe to a second probe while hybridized on a monomorphic target polynucleotide, wherein the first positive control probe of the specific ligation product differs from the first negative control probe in the non-specific ligation product only by a discriminating region, wherein the monomorphic target polynucleotide queried by the first positive control probe is the same as the monomorphic target polynucleotide queried by the first negative control probe; quantifying the difference between the amount of the specific positive control ligation product and the non-specific negative control ligation product, thereby determining ligation specificity in a ligation assay.

In some embodiments, the methods of the present teachings further comprise comparing the amount of an experimental ligation product to the amount of the specific positive control ligation product and the amount of the non-specific negative control ligation product, wherein the experimental ligation product, the specific positive control ligation product, and the non-specific negative control ligation product are derived from the same ligation reaction, and determining therefrom the ligation specificity for the experimental ligation product in a ligation assay.

In some embodiments the variability of specific ligation in parallel ligation assays are assessed comprising; comparing the amount of a specific positive control ligation product in a first reaction to a specific positive control ligation product in a second reaction, wherein the specific positive control ligation product in the first reaction results from a positive control probe set, wherein the positive control probe set comprises a first positive control probe and a second probe, wherein the specific positive control ligation product in the second reaction results from a positive control probe set, wherein the positive control probe set comprises a first positive control probe and a second probe, wherein the first positive control probe of the first reaction does not differ from the first positive control probe in the second reaction, wherein the second probe of the first reaction does not differ from the second probe of the second reaction, wherein a monomorphic target polynucleotide queried by the positive control probe set in the first reaction is the same as a monomorphic target polynucleotide queried by the positive control probe set in the second reaction; quantifying the difference between the amount of the specific ligation product in the first reaction and the amount of the specific ligation product in the second reaction, thereby assessing the variability of specific ligation in a ligation assay.

In some embodiments, the first reaction comprises a plurality of positive control probe sets, wherein each positive control probe set in the first reaction queries a different monomorphic target polynucleotide, wherein the second reaction comprises a plurality of positive control probe sets, wherein each positive control probe set in the second reaction queries a different monomorphic target polynucleotide, wherein the monomorphic target polynucleotides queried in the first reaction are the same as the monomorphic target polynucleotides queried in the second reaction, wherein the positive control first probe of the positive control probe set querying a given monomorphic target polynucleotide in the first reaction comprises an identifying portion, wherein the positive control first probe of the positive control set querying a given monomorphic target polynucleotide in the second reaction comprises an identifying portion, wherein the identifying portion of the positive control first probe in the first reaction querying a given monomorphic target polynucleotide is the same as the identifying portion of the positive control first probe in the second reaction querying that same monomorphic target polynucleotide.

In some embodiments of the present teachings each positive control probe set comprises a first positive control probe one and a first positive control probe two, wherein the first positive control probe one and the first positive control probe two each comprise an identifying portion, wherein the identifying portion of the first positive control probe one differs from the identifying portion of first positive probe two, wherein the identifying protion of the positive control first probe one querying a given monomorphic target polynucleotide in the first reaction is the same as the identifying portion of the positive control first probe one querying that same monomorphic target polynucleotide in the second reaction, wherein the identifying protion of the positive control first probe two querying a given monomorphic target polynucleotide in the first reaction is the same as the identifying portion of the positive control first probe two querying that same monomorphic target polynucleotide in the second reaction.

In some embodiments, the present teachings provide a method for assessing the variability of non-specific ligation in parallel ligation assays comprising; comparing the amount of a non-specific negative control ligation product in a first reaction to a non-specific negative control ligation product in a second reaction, wherein the non-specific negative control ligation product in the first reaction results from a negative control probe set, wherein the negative control probe set comprises a first negative control probe and a second probe, wherein the non-specific negative control ligation product in the second reaction results from a negative control probe set, wherein the negative control probe set comprises a first negative control probe and a second probe, wherein the first negative control probe of the first reaction does not differ from the first negative control probe in the second reaction, wherein the second probe of the first reaction does not differ from the second probe of the second reaction, wherein a monomorphic target polynucleotide queried by the negative control probe set in the first reaction is the same as a monomorphic target polynucleotide queried by the negative control probe set in the second reaction; quantifying the difference between the amount of the non-specific ligation product in the first reaction and the amount of non-specific ligation product in the second reaction, thereby assessing the variability of non-specific ligation in a ligation assay.

In some embodiments, the first reaction comprises a plurality of negative control probe sets, wherein each negative control probe set in the first reaction queries a different monomorphic target polynucleotide, wherein the second reaction comprises a plurality of negative control probe sets, wherein each negative control probe set in the second reaction queries a different monomorphic target polynucleotide, wherein the monomorphic target polynucleotides queried in the first reaction are the same as the monomorphic target polynucleotides queried in the second reaction, wherein the negative control first probe of the negative control probe set querying a given monomorphic target polynucleotide in the first reaction comprises an identifying portion, wherein the negative control first probe of the negative control set querying a given monomorphic target polynucleotide in the second reaction comprises an identifying portion, wherein the identifying portion of the negative control first probe in the first reaction querying a given monomorphic target polynucleotide is the same as the identifying portion of the negative control first probe in the second reaction querying that same monomorphic target polynucleotide.

In some embodiments each negative control probe set comprises a first negative control probe one and a first negative control probe two, wherein the first negative control probe one and the first negative control probe two each comprise an identifying portion, wherein the identifying portion of first negative control probe one differs from the identifying portion of the first negative probe two, wherein the identifying protion of the negative control first probe one querying a given monomorphic target polynucleotide in the first reaction is the same as the identifying portion of the negative control first probe one querying that same monomorphic target polynucleotide in the second reaction, wherein the identifying protion of the negative control first probe two querying a given monomorphic target polynucleotide in the first reaction is the same as the identifying portion of the negative control first probe two querying that same monomorphic target polynucleotide in the second reaction.

In some embodiments, the present teachings provide a method for assessing the variability of non-specific and specific ligation in parallel ligation assays comprising; comparing the amount of a non-specific negative control ligation product in a first reaction to a specific control ligation product in a second reaction, wherein the non-specific negative control ligation product in the first reaction results from a negative control probe set, wherein the negative control probe set comprises a first negative control probe and a second probe, wherein the specific positive control ligation product in the second reaction results from a positive control probe set, wherein the positive control probe set comprises a first positive control probe and a second probe, wherein the first negative control probe of the first reaction differs from the first positive control probe in the second reaction by only a discriminating region, wherein the second probe of the first reaction does not differ from the second probe of the second reaction, wherein a monomorphic target polynucleotide queried by the negative control probe set in the first reaction is the same as a monomorphic target polynucleotide queried by the positive control probe set in the second reaction; quantifying the difference between the amount of the non-specific ligation product in the first reaction and the amount of specific ligation product in the second reaction, thereby assessing the variability of specific and non-specific ligation in a ligation assay.

In some embodiments, the first reaction comprises a plurality of negative control probe sets, wherein each negative control probe set in the first reaction queries a different monomorphic target polynucleotide, wherein the second reaction comprises a plurality of positive control probe sets, wherein each positive control probe set in the second reaction queries a different monomorphic target polynucleotide, wherein the monomorphic target polynucleotides queried in the first reaction are the same as the monomorphic target polynucleotides queried in the second reaction, wherein the negative control first probe of the negative control probe set querying a given monomorphic target polynucleotide in the first reaction comprises an identifying portion, wherein the negative control first probe of the positive control set querying a given monomorphic target polynucleotide in the second reaction comprises an identifying portion, wherein the identifying portion of the negative control first probe in the first reaction querying a given monomorphic target polynucleotide is the same as the identifying portion of the positive control first probe in the second reaction querying that same monomorphic target polynucleotide.

In some embodiments each negative control probe set in the first reaction comprises a first negative control probe one and a first negative control probe two, wherein the first negative control probe one and the first negative control probe two each comprise an identifying portion, wherein the identifying portion of first negative control probe one differs from the identifying portion of the first negative probe two, wherein each positive control probe set in the second reaction comprises a first positive control probe one and a first positive control probe two, wherein the first positive control probe one and the first positive control probe two each comprise an identifying portion, wherein the identifying portion of the positive control first probe one differs from the identifying portion of the first positive probe two, wherein the identifying portion of the negative control first probe one querying a given monomorphic target polynucleotide in the first reaction is the same as the identifying portion of the positive control first probe one querying that same monomorphic target polynucleotide in the second reaction, wherein the identifying protion of the negative control first probe two querying a given monomorphic target polynucleotide in the first reaction is the same as the identifying portion of the positive control first probe two querying that same monomorphic target polynucleotide in the second reaction.

In some embodiments, the present teachings provide a kit for assessing ligation comprising a positive control probe set and a negative control probe set, an experimental probe set, and combinations thereof.

In some embodiments, the present teachings provide a kit for assessing ligation comprising a plurality of positive control probe sets.

In some embodiments, the present teachings provide a kit of assessing ligation comprising a plurality of negative control probe sets.

In some embodiments, the kits of the present teachings further comprise a plurality of monomorphic target polynucleotides, a plurality of polymorphic target polynucleotides, a means for ligating, a means for phosphorylating, a means for amplifying, and combinations thereof.

In some embodiments, the present teachings provide a method of determining ligation specificity comprising the steps of, hybridizing, ligating, amplifying, removing, separating, detecting, comparing, and determining therefrom ligation specificity.

### BRIEF DESCRIPTION OF THE DRAWINGS

FIG. 1 depicts some method embodiments of the present teachings.

FIG. 2 depicts some method embodiments of the present teachings.

FIG. 3 depicts some method embodiments of the present teachings.

FIG. 4 depicts some method embodiments of the present teachings.

FIG. 5 depicts some method embodiments of the present teachings.

FIG. 6 depicts some method embodiments of the present teachings.

FIG. 7 depicts some composition useful for some of the method embodiments of the present teachings.

FIG. 8 depicts some method embodiments of the present teachings.

### DESCRIPTION OF EXEMPLARY EMBODIMENTS

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control.

It is to be understood that both the foregoing general description and the following detailed description are exemplary and explanatory only and are not restrictive of the invention. In this application, the use of the singular includes the plural unless specifically stated otherwise. For example, “a probe” means that more than one probe can be present; for example, one or more copies of a particular probe species, as well as one or more versions of a particular probe type. Also, the use of “or” means “and/or” unless stated otherwise. Similarly, “comprise”, “comprises”, “comprising”, “include”, “includes”, and “including” are not intended to be limiting.

### CERTAIN DEFINITIONS

As used herein, the “probes,” “primers,” “targets,” “oligonucleotides,” “polynucleotides,” “nucleobase sequences,” and “oligomers” of the present teachings can be comprised of at least one of ribonucleotides, deoxynucleotides, modified ribonucleotides, modified deoxyribonucleotides, modified phosphate-sugar-backbone oligonucleotides, nucleotide analogs, and combinations thereof, and can be single stranded, double stranded, or contain portions of both double stranded and single stranded sequence, as appropriate. Some more elaborative and non-limiting definitions are provided infra.

The term “nucleotide”, as used herein, generically encompasses the following terms, which are defined below: nucleotide base, nucleoside, nucleotide analog, extendable, and universal nucleotide.

The term “nucleotide base”, as used herein, refers to a substituted or unsubstituted parent aromatic ring or rings. In some embodiments, the aromatic ring or rings contain at least one nitrogen atom. In some embodiments, the nucleotide base is capable of forming Watson-Crick and/or Hoogsteen hydrogen bonds with an appropriately complementary nucleotide base. Exemplary nucleotide bases and analogs thereof include, but are not limited to, purines such as 2-aminopurine, 2,6-diaminopurine, adenine (A), ethenoadenine, N6-Δ2-isopentenyladenine (6iA), N6-Δ2-isopentenyl-2-methylthioadenine (2 ms6iA), N6-methyladenine, guanine (G), isoguanine, N2-dimethylguanine (dmG), 7-methylguanine (7mG), 2-thiopyrimidine, 6-thioguanine (6sG) hypoxanthine and 06-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, 04-methylthymine, uracil (U), 4-thiouracil (4sU) and 5,6-dihydrouracil (dihydrouracil; D); indoles such as nitroindole and 4-methylindole; pyrroles such as nitropyrrole; nebularine; base (Y); etc. In some embodiments, nucleotide bases are universal nucleotide bases. Additional exemplary nucleotide bases can be found, e.g., in Fasman, 1989, Practical Handbook of Biochemistry and Molecular Biology, pp. 385-394, CRC Press, Boca Raton, Fla., and the references cited therein. Further examples of universal bases can be found for example in Loakes, N. A. R. 2001, vol 29:2437-2447 and Seela N. A. R. 2000, vol 28:3224-3232.

The term “nucleoside”, as used herein, refers to a compound having a nucleotide base covalently linked to the C-1′ carbon of a pentose sugar. In some embodiments, the linkage is via a heteroaromatic ring nitrogen. Typical pentose sugars include, but are not limited to, those pentoses in which one or more of the carbon atoms are each independently substituted with one or more of the same or different —R, —OR, —NRR or halogen groups, where each R is independently hydrogen, (C1-C6) alkyl or (C5-C14) aryl. The pentose sugar may be saturated or unsaturated. Exemplary pentose sugars and analogs thereof include, but are not limited to, ribose, 2′-deoxyribose, 2′-(C1-C6)alkoxyribose, 2′-(C5-C14)aryloxyribose, 2′,3′-dideoxyribose, 2′,3′-didehydroribose, 2′-deoxy-3′-haloribose, 2′-deoxy-3′-fluororibose, 2′-deoxy-3′-chlororibose, 2′-deoxy-3′-α-minoribose, 2′-deoxy-3′-(C1-C6) alkylribose, 2′-deoxy-3′-(C1-C6)alkoxyribose and 2′-deoxy-3′-(C5-C14)aryloxyribose. Also see e.g. 2′-O-methyl, 4′-α-anomeric nucleotides, 1′-α-anomeric nucleotides (Asseline (1991) Nucl. Acids Res. 19:4067-74), 2′-4′- and 3′-4′linked and other “locked” or “LNA”, bicyclic sugar modifications (WO 98/22489; WO 98/39352; WO 99/14226). “LNA” or “locked nucleic acid” is a DNA analogue that is conformationally locked such that the ribose ring is constrained by a methylene linkage between the 2′-oxygen and the 3′- or 4′-carbon. The conformation restriction imposed by the linkage often increases binding affinity for complementary sequences and increases the thermal stability of such duplexes.

Exemplary LNA sugar analogs within a polynucleotide include the structures: 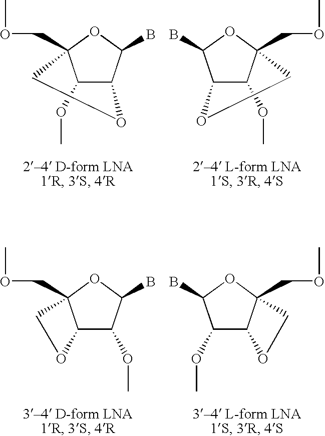
where B is any nucleobase.

Sugars include modifications at the 2′- or 3′-position such as methoxy, ethoxy, allyloxy, isopropoxy, butoxy, isobutoxy, methoxyethyl, alkoxy, phenoxy, azido, amino, alkylamino, fluoro, chloro and bromo. Nucleosides and nucleotides include the natural D configurational isomer (D-form), as well as the L configurational isomer (L-form) (Beigelman, U.S. Pat. No. 6,251,666; Chu, U.S. Pat. No. 5,753,789; Shudo, EP0540742; Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleobase is purine, e.g. A or G, the ribose sugar is attached to the N⁹-position of the nucleobase. When the nucleobase is pyrimidine, e.g. C, T or U, the pentose sugar is attached to the N¹-position of the nucleobase (Kornberg and Baker, (1992) DNA Replication, 2^{nd} Ed., Freeman, San Francisco, Calif.).

One or more of the pentose carbons of a nucleoside may be substituted with a phosphate ester having the formula: 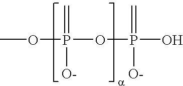
where α is an integer from 0 to 4. In some embodiments, α is 2 and the phosphate ester is attached to the 3′- or 5′-carbon of the pentose. In some embodiments, the nucleosides are those in which the nucleotide base is a purine, a 7-deazapurine, a pyrimidine, a universal nucleotide base, a specific nucleotide base, or an analog thereof.

The term “nucleotide analog”, as used herein, refers to embodiments in which the pentose sugar and/or the nucleotide base and/or one or more of the phosphate esters of a nucleoside may be replaced with its respective analog. In some embodiments, exemplary pentose sugar analogs are those described above. In some embodiments, the nucleotide analogs have a nucleotide base analog as described above. In some embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions. Other nucleic acid analogs and bases include for example intercalating nucleic acids (INAs, as described in Christensen and Pedersen, 2002), and AEGIS bases (Eragen, U.S. Pat. No. 5,432,272). Additional descriptions of various nucleic acid analogs can also be found for example in (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 1 1 0:4470 (1988); and Pauwels et al., Chemica Scripta 26:141 91986)), phosphorothioate (Mag et al., Nucleic Acids Res. 19:1437 (1991); and U.S. Pat. No. 5,644,048. Other nucleic analogs comprise phosphorodithioates (Briu et al., J. Am. Chem. Soc. 11 1:2321 (1989), 0-methylphophoroamidite linkages (see Eckstein, Oligonucleotides and Analogues: A Practical Approach, Oxford University Press), those with positive backbones (Denpcy et al., Proc. Natl. Acad. Sci. USA 92:6097 (1995); non-ionic backbones (U.S. Pat. Nos. 5,386,023, 5,386,023, 5,637,684, 5,602,240, 5,216,141, and 4,469,863. Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1 9 4): Chaq.ters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biornolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) ppl 69-176). Several nucleic acid analogs are also described in Rawls, C & E News Jun. 2, 1997 page 35.

The term “universal nucleotide base” or “universal base”, as used herein, refers to an aromatic ring moiety, which may or may not contain nitrogen atoms. In some embodiments, a universal base may be covalently attached to the C-1′ carbon of a pentose sugar to make a universal nucleotide. In some embodiments, a universal nucleotide base does not hydrogen bond specifically with another nucleotide base. In some embodiments, a universal base hydrogen bonds with a nucleotide base, up to and including all nucleotide bases in a particular target polynucleotide. In some embodiments, a nucleotide base may interact with adjacent nucleotide bases on the same nucleic acid strand by hydrophobic stacking. Universal nucleotides include, but are not limited to, deoxy-7-azaindole triphosphate (d7AITP), deoxyisocarbostyril triphosphate (dICSTP), deoxypropynylisocarbostyril triphosphate (dPICSTP), deoxymethyl-7-azaindole triphosphate (dM7AITP), deoxylmPy triphosphate (dlmPyTP), deoxyPP triphosphate (dPPTP), or deoxypropynyl-7-azaindole triphosphate (dP7AITP). Further examples of such universal bases can be found, inter alia, in Published U.S. application Ser. No. 10/290,672, and U.S. Pat. No. 6,433,134.

As used herein, the terms “polynucleotide” and “oligonucleotide” are used interchangeably and mean single-stranded and double-stranded polymers of nucleotide monomers, including 2′-deoxyribonucleotides (DNA) and ribonucleotides (RNA) linked by internucleotide phosphodiester bond linkages, e.g. 3′-5′ and 2′-5′, inverted linkages, e.g. 3′-3′ and 5′-5′, branched structures, or internucleotide analogs. Polynucleotides have associated counter ions, such as H⁺, NH₄⁺, trialkylammonium, Mg²⁺, Na⁺ and the like. A polynucleotide may be composed entirely of deoxyribonucleotides, entirely of ribonucleotides, or chimeric mixtures thereof. Polynucleotides may be comprised of internucleotide, nucleobase and/or sugar analogs. Polynucleotides typically range in size from a few monomeric units, e.g. 3-40 when they are more commonly frequently referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine, “C” denotes deoxycytosine, “G” denotes deoxyguanosine, and “T” denotes thymidine, unless otherwise noted.

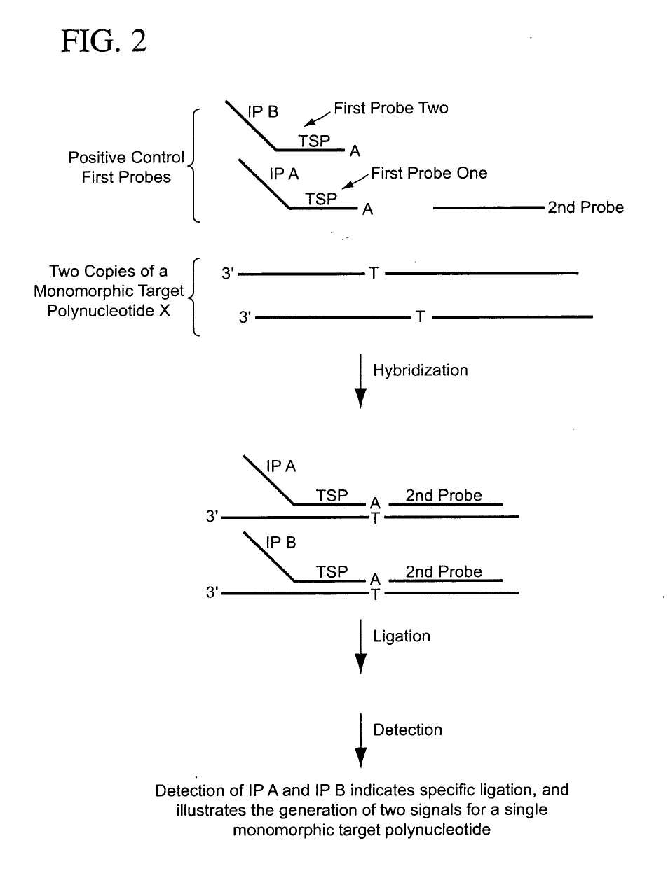

As used herein, “nucleobase” means those naturally occurring and those non-naturally occurring heterocyclic moieties commonly known to those who utilize nucleic acid technology or utilize peptide nucleic acid technology to thereby generate polymers that can sequence specifically bind to nucleic acids. Non-limiting examples of suitable nucleobases include: adenine, cytosine, guanine, thymine, uracil, 5-propynyl-uracil, 2-thio-5-propynyl-uracil, 5-methlylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). Other non-limiting examples of suitable nucleobase include those nucleobases illustrated in FIGS. 2(A) and 2(B) of Buchardt et al. (WO92/20702 or WO92/20703).

As used herein, “nucleobase sequence” means any segment, or aggregate of two or more segments (e.g. the aggregate nucleobase sequence of two or more oligomer blocks), of a polymer that comprises nucleobase-containing subunits. Non-limiting examples of suitable polymers or polymers segments include oligodeoxynucleotides (e.g. DNA), oligoribonucleotides (e.g. RNA), peptide nucleic acids (PNA), PNA chimeras, PNA combination oligomers, nucleic acid analogs and/or nucleic acid mimics.

As used herein, “polynucleobase strand” means a complete single polymer strand comprising nucleobase subunits. For example, a single nucleic acid strand of a double stranded nucleic acid is a polynucleobase strand.

As used herein, “nucleic acid” is a nucleobase sequence-containing polymer, or polymer segment, having a backbone formed from nucleotides, or analogs thereof. Preferred nucleic acids are DNA and RNA.

As used herein, “peptide nucleic acid” or “PNA” means any oligomer or polymer segment (e.g. block oligomer) comprising two or more PNA subunits (residues), but not nucleic acid subunits (or analogs thereof, including, but not limited to, any of the oligomer or polymer segments referred to or claimed as peptide nucleic acids in U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, and 6,107,470. The term PNA shall also apply to any oligomer or polymer segment comprising two or more subunits of those nucleic acid mimics described in the following publications: Lagriffoul et al., Bioorganic & Medicinal Chemistry Letters, 4: 1081-1082 (1994); Petersen et al., Bioorganic & Medicinal Chemistry Letters, 6: 793-796 (1996); Diderichsen et al., Tett. Lett. 37: 475-478 (1996); Fujii et al., Bioorg. Med. Chem. Lett. 7: 637-627 (1997); Jordan et al., Bioorg. Med. Chem. Lett. 7: 687-690 (1997); Krotz et al., Tett. Lett. 36: 6941-6944 (1995); Lagriffoul et al., Bioorg. Med. Chem. Lett. 4: 1081-1082 (1994); Diederichsen, U., Bioorganic & Medicinal Chemistry Letters, 7: 1743-1746 (1997); Lowe et al., J. Chem. Soc. Perkin Trans. 1, (1997) 1: 539-546; Lowe et J. Chem. Soc. Perkin Trans. 11: 547-554 (1997); Lowe et al., J. Chem. Soc. Perkin Trans. 1 1:5 55-560 (1997); Howarth et al., J. Org. Chem. 62: 5441-5450 (1997); Altmann, K-H et al., Bioorganic & Medicinal Chemistry Letters, 7: 1119-1122 (1997); Diederichsen, U., Bioorganic & Med. Chem. Lett, 8: 165-168 (1998); Diederichsen et al., Angew. Chem. Int Ed., 37: 302-305 (1998); Cantin et al., Tett. Lett., 38: 4211-4214 (1997); Ciapetti et al., Tetrahedron, 53: 1167-1176 (1997); Lagriffoule et al., Chem. Eur. J., 3: 912-919 (1997); Kumar et al., Organic Letters 3(9): 1269-1272 (2001); and the Peptide-Based Nucleic Acid Mimics (PENAMS) of Shah et al. as disclosed in WO96/04000.

In some embodiments, PNA is an oligomer or polymer segment comprising two or more covalently linked subunits of the formula found in paragraph 76 of U.S. Patent Application 2003/0077608A1 wherein, each J is the same or different and is selected from the group consisting of H, R¹, OR¹, SR¹, NHR¹, NR¹₂, F, Cl, Br and I. Each K is the same or different and is selected from the group consisting of O, S, NH and NR¹. Each R¹ is the same or different and is an alkyl group having one to five carbon atoms that may optionally contain a heteroatom or a substituted or unsubstituted aryl group. Each A is selected from the group consisting of a single bond, a group of the formula; (CJ₂)ₛ- and a group of the formula; —(CJ₂)ₙC(O)—, wherein, J is defined above and each s is a whole number from one to five. Each t is 1 or 2 and each u is 1 or 2. Each L is the same or different and is independently selected from: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine), other naturally occurring nucleobase analogs or other non-naturally occurring nucleobases.

In some other embodiments, a PNA subunit comprises a naturally occurring or non-naturally occurring nucleobase attached to the N-α-glycine nitrogen of the N-[2-(aminoethyl)]glycine backbone through a methylene carbonyl linkage; this currently being the most commonly used form of a peptide nucleic acid subunit.

As used herein, “target polynucleotide sequence” is a nucleobase sequence of a polynucleobase strand sought to be determined. It is to be understood that the nature of the target sequence is not a limitation of this invention. The polynucleobase strand comprising the target sequence may be provided from any source. For example, the target sequence may exist as part of a nucleic acid (e.g. DNA or RNA), PNA, nucleic acid analog or other nucleic acid mimic. The target can be methylated, non-methylated, or both. The sample containing the target sequence may be from any source, and is not a limitation of the present teachings. Further, it will be appreciated that “target” can refer to both a “target polynucleotide sequence” as well as surrogates thereof, for example ligation products, amplification products, and sequences encoded therein.

As used herein, the term “primer portion” refers to a region of a polynucleotide sequence that can serve directly, or by virtue of its complement, as the template upon which a primer can anneal for any of a variety of primer nucleotide extension reactions known in the art (for example, PCR). It will be appreciated by those of skill in the art that when two primer portions are present on a single polynucleotide (for example an OLA product, a PCR product, etc), the orientation of the two primer portions is generally different. For example, one PCR primer can directly hybridize to the first primer portion, while the other PCR primer can hybridize to the complement of the second primer portion. Stated another way, the first primer portion can be in a sense orientation, and the second primer portion can be in an antisense orientation. In addition, “universal” primers and primer portions as used herein are generally chosen to be as unique as possible given the particular assays and host genomes to ensure specificity of the assay. However, as will be appreciated by those of skill in the art, different configurations of primer portions can be used, for example one reaction can utilize 500 first probes with a first primer portion or battery of primer portions, and an additional 500 second probes with a second primer portion or battery of primer portions. Further, all of the universal primer portions can be the same for all targets in a reaction thereby allowing, for example, a single upstream primer and a single downstream primer to amplify all targets, and/or, a single primer to serve as both upstream and downstream primer to amplify all targets. Alternatively, “batteries” of universal upstream primer portions and batteries of universal downstream primer portions can used, either simultaneously or sequentially. In some embodiments, at least one of the primer portions can comprise a T7 RNA polymerase site.

As used herein, “forward” and “reverse” are used to indicate relative orientation of probes on a target, and generally refer to a 5′ to 3′ “forward” oriented primer hybridized to the 3′ end of the ‘top’ strand of a target polynucleotide, and a 5′ to 3′ “reverse” oriented primer hybridized to the 3′ end of the bottom strand of a target polynucleotide. As will be recognized by those of skill in the art, these terms are not-intended to be limiting, but rather provide illustrative orientation in any given embodiment.

As used herein, the term “sample” refers to a mixture from which the at least one target polynucleotide sequence is derived, such sources including, but not limited to, raw viruses, prokaryotes, protists, eukaryotes, plants, fungi, and animals. These sample sources may include, but are not limited to, whole blood, a tissue biopsy, lymph, bone marrow, amniotic fluid, hair, skin, semen, biowarfare agents, anal secretions, vaginal secretions, perspiration, various environmental samples (for example, agricultural, water, and soil), research samples generally, purified samples generally, and cultured cells. It will be appreciated that nucleic acids can be isolated from samples using any of a variety of procedures known in the art, for example the Applied Biosystems ABI Prism TM 6100 Nucleic Acid PrepStation, and the ABI Prism TM 6700 Automated Nucleic Acid Workstation, Boom et al., U.S. Pat. No. 5,234,809, etc. It will be appreciated that nucleic acids can be cut or sheared prior to analysis, including the use of such procedures as mechanical force, sonication, restriction endonuclease cleavage, or any method known in the art.

It will be appreciated that the selection of the probes to query a given target polynucleotide sequence, and the selection of which target polynucleotide sequences to collect in a given reaction, will involve procedures generally known in the art, and can involve the use of algorithms to select for those sequences with minimal secondary and tertiary structure, those targets with minimal sequence redundancy with other regions of the genome, those target regions with desirable thermodynamic characteristics, and other parameters desirable for the context at hand. In some embodiments, probes can further comprise various modifications such as a minor groove binder (see for example U.S. Pat. No. 6,486,308) to further provide desirable thermodynamic characteristics.

As used herein, the term “monomorphic target polynucleotide sequence” refers to a nucleobase sequence in which all of the copies of the sequence in the reaction are believed to comprise the same sequence of nucleobases (that is, the monomorphic target polynucleotide sequence is believed to lack any polymorphic bases). In some embodiments, the monomorphic target polynucleotide sequence can comprise a genomic locus, though it will be appreciated that any nucleobase sequence can serve as a monomorphic target polynucleotide sequence. Monomorphic target sequences can be acquired in the following way: 1,000 s of putative (candidate) SNPs can be sequenced against dozens of different genomic DNAs (for example human DNA). Putative SNP loci not showing any polymorphisms can be considered “false” or low minor allele frequency, and can subsequently be used as monomorphic targets polynucleotides. Two sample sequences produced in this fashion include:

PC1004683 (SEQUENCE ID NO:1)
CTCCATCTCCTCCACTGTTCCCCCACACTGTGCTGTGACA[A/A]TGAGATGAGACAG
AGGGTCAGGACAACATCAAGGGGTGTA
PC1004706 (SEQUENCE ID NO:2)
AAAGACATAAACCTCCCTGTGACTCCATTTTGGTAACTGT[A/A]TCCAAAACACAGGA
TCCCTGCTGTTCTTTGTTTCCTTTTA

As used herein, the term “probe set” refers to at least one first probe and at least one second probe that together query a given target polynucleotide sequence.

For example, a “positive control probe set” comprises at least one positive control first probe and at least one positive control second probes, which can query a given monomorphic target polynucleotide sequence, wherein positive control first probes of a given positive control probe set differ only in their identifying portions and comprise the same target specific portions. When a primer portion is present, in some embodiments all primer portions for the first probes in a reaction can be the same, and all primer portions for the second probes in a reaction can be the same, though it will be appreciated they need not be.

For example, a “negative control probe set” comprises at least one negative control first probe and at least one negative control second probe, which can query a given monomorphic target polynucleotide sequence, wherein negative control first probes of given negative control set differ only in their identifying portions and comprise the same target specific portions. When a primer portion is present, in some embodiments all primer portions for the first probes in a reaction can be the same, and all primer portions for the second probes in a reaction can be the same, though it will be appreciated they need not be.

For example, an “experimental probe set” comprises at least one experimental first probe and at least one experimental second probe, which can query a given target polymorphic target polynucleotide sequence, wherein experimental first probes of a given experimental probe set differ in the discriminating region of the target specific portion, as well as in their target identifying portion. When a primer portion is present, in some embodiments all primer portions for the first probes in a reaction can be the same, and all primer portions for the second probes in a reaction can be the same, though it will be appreciated they need not be.

As used herein, the term “first probe” refers generally to at least one oligonucleotide that can hybridize to a target polynucleotide sequence adjacent to a second probe, and that generally comprises a target specific portion, wherein the target specific portion comprises a disciminating region, a target identifying portion, and optionally a primer portion. In some embodiments, “positive control first probes” can hybridize to a target monomorphic polynucleotide. When positive control first probes are hybridized adjacent and contiguous to a “positive control second probe,” specific ligation can occur. When more than one positive control first probes are present in a set, the positive control first probes can differ in their identifying portion, and are referred to as “positive control first probe 1, positive control first probe 2, etc. In some embodiments, “negative control first probes” can hybridize to a target monomorphic polynucleotide. When negative control first probes are hybridized adjacent and contiguous to a “negative control second probe,” specific ligation does not occur, but non-specific ligation can occur. When more than one negative control first probes are present in a set, the negative control first probes can differ in their identifying portion, and are referred to as negative control first probe 1, negative control first probe 2, etc. In some embodiments, “experimental first probes” can hybridize to a target polymorphic polynucleotide. When experimental first probes are hybridized adjacent and contiguous to a “experimental second probe,” specific ligation can occur. When more than one experimental first probes are present in a set, the experimental first probes can differ in their discriminating nucleotide and in their identifying portion, and are referred to as experimental first probe 1, experimental first probe 2, etc In some embodiments, the first probes are located 5′ (that is, upstream) to the second probe, and the first probes and second probes hybridize to adjacent regions of the same target polynucleotide sequence. In some embodiments, the first probes can hybridize to the target polynucleotide sequence in the absence of any second probe in the reaction, for example, control first probes need not be hybridized to an adjacent second control probe, but can nonetheless be considered control first probes. It will be appreciated that the terms “upstream” and “downstream” are terms to orient the reader given a particular embodiment of the present teachings, and that for example a first probe can be located 3′ (that is, downstream) to the second probes, for example when the 5′ to 3′ orientation of the target is switched, and that such is clearly contemplated by the present teachings. Further, it will be appreciated that the target polynucleotide can be either of the strands of a double stranded polynucleotide. As used herein, the identifying portion of a given probe will be notated with a capital letter, for example “A” or “B,” which is intended to convey that the identifying portions of the probes at issue are distinguishable and different from one another.

As used herein, the term “second probe” refers generally to at least one oligonucleotide that can hybridize to a target polynucleotide sequence adjacent to a first probe, and that generally comprises a target specific portion and optionally a primer portion.

As used herein, the term “same” will be recognized to mean the same nucleobase sequence rather than the same molecule. For example, it will be appreciated that the target polynucleotide sequences of the present teachings can be present in multiple copies in a given reaction. The same sequence in this context refers to the same sequence of nucleobases, rather than the same molecule.

As used herein, the term “or combinations thereof” can refer to all permutations and combinations of the listed items preceding the term. For example, “X, Y, Z, or combinations thereof” is intended to include at least one of: X, Y, Z, XY, XZ, YZ, or XYZ, and if order is important in a particular context, also YX, ZX, ZY, ZYX, YZX, or ZXY. Continuing with this example, expressly included are combinations that contain repeats of one or more item or term, such as YY, XXX, XXY, YYZ, XXXYZZZZ, ZYYXXX, ZXYXYY, and so forth. The skilled artisan will understand that typically there is no limit on the number of items or terms in any combination, unless otherwise apparent from the context.

As used herein, the term “target specific portion” refers to the portion of a probe substantially complementary to a target polynucleotide sequence, and can further comprise a discriminating region.

The term “corresponding” as used herein refers to at least one specific relationship between the elements to which the term refers. For example, at least one first probe of a probe set corresponds to at least one second probe of the same probe set, and vice versa. At least one primer is designed to anneal with the primer portion of at least one corresponding probe, at least one corresponding ligation product, at least one corresponding amplified ligation product, or combinations thereof. The target-specific portions of the probes of a particular probe set can be designed to hybridize with a complementary or substantially complementary region of the corresponding target polynucleotide sequence. A particular affinity moiety can bind to the corresponding affinity moiety binder, for example but not limited to, the affinity moiety binder streptavidin binding to the affinity moiety biotin. A particular mobility probe can hybridize with the corresponding identifier portion or identifying portion complement. A particular discriminating region can hybridize to the corresponding nucleotide or nucleotides on the target polynucleotide, as so forth.

As used herein the term “contiguous” refers to the absence of a gap between the terminal nucleobase of at least two adjacently hybridized oligonucleotides, such that the at least two oligonucleotides are abutting one another and are potentially suitable for ligation.

As used herein the term “parallel reaction” refers generally to at least two reactions occurring roughly at the same time, but in different reaction vessels. For example, two different wells in a microtitre plate can comprise parallel reactions, though it will be appreciated that parallel reactions can occur at different periods of time, and/or in different instruments or geographical places, and still be considered parallel reactions for the purposes of the present teachings.

As used herein, the term “discriminating region” refers generally to that region of the target specific portion of a first probe that can, or cannot, be complementary with a corresponding region of the target polynucleotide sequence. In some embodiments, the discriminating nucleotide is located at the 3′ end of the target specific portion of a first probe, though it will be appreciated that the discriminating region can be in other regions of the first probe as well. It will be appreciated that the discriminating region can refer to a single nucleotide, or more than one single nucleotide. In the case of positive control first probes, the discriminating region will in general be complementary to the monomorphic target polynucleotide. In the case of negative control first probes, the discriminating region will in general not be complementary to the monomorphic target polynucleotide. In the case of experimental first probes, the discriminating region of first probes can in general query different versions of a polymorphic target polynucleotide. Further, in the case of experimental first probes, the discriminating region of a first probe one can differ from the discriminating region of a first probe two, which can be indicated by referring to a “discriminating region one” of a first probe one, and a “discriminating region two” of a first probe two.

As used herein, the term “polymorphic target polynucleotide sequence” refers to a nucleobase sequence believed to potentially comprise at least one nucleobase variant sequence (that is, the polymorphic target polynucleotide sequence is believed to potentially comprise at least one polymorphic nucleobase). In some embodiments, the polymorphic target polynucleotide sequence can comprise a genomic locus wherein the variant nucleobase corresponds with a particular allelic variant of a SNP locus, thereby resulting in a heterozygotic polymorphic target polynucleotide sequence, though it will be appreciated that any variant in the nucleobase sequence can provide a polymorphic target polynucleotide sequence. Further, it will be appreciated that the putative nucleobase variant need not vary, in such manner for example as with a homozygote. It will be appreciated that polymorphic target polynucleotides of the present teachings can comprise methylated nucleic acids, and optionally, bisulfite-treated nucleic acids wherein non-methylated cytosines are converted into thymine. Further, it will be appreciated that target polymorphic polynucleotides of the present teachings can further comprise mRNA, and/or cDNA versions therof, including various splice variants of a given gene.

As used herein the terms “annealing” and “hybridization” are used interchangeably and mean the complementary base-pairing interaction of one nucleic acid with another nucleic acid that results in formation of a duplex, triplex, or other higher-ordered structure. In some embodiments, the primary interaction is base specific, e.g., A/T and G/C, by Watson/Crick and Hoogsteen-type hydrogen bonding. In some embodiments, base-stacking and hydrophobic interactions may also contribute to duplex stability. Conditions for hybridizing nucleic acid probes and primers to complementary and substantially complementary target sequences are well known, e.g., as described in Nucleic Acid Hybridization, A Practical Approach, B. Hames and S. Higgins, eds., IRL Press, Washington, D.C. (1985) and J. Wetmur and N. Davidson, Mol. Biol. 31:349 et seq. (1968). In general, whether such annealing takes place is influenced by, among other things, the length of the probes and the complementary target sequences, the pH, the temperature, the presence of mono- and divalent cations, the proportion of G and C nucleotides in the hybridizing region, the viscosity of the medium, and the presence of denaturants. Such variables influence the time required for hybridization. Thus, the preferred annealing conditions will depend upon the particular application. Such conditions, however, can be routinely determined by the person of ordinary skill in the art without undue experimentation. Further, in general probes and primers of the present teachings are designed to be complementary to a target sequence, such that hybridization of the target and the probes or primers occurs. It will be appreciated, however, that this complementarity need not be perfect; there can be any number of base pair mismatches that will interfere with hybridization between the target sequence and the single stranded nucleic acids of the present teachings. However, if the number of base pair mismatches is so great that no hybridization can occur under even the least stringent of hybridization conditions, the sequence is not a complementary target sequence. Thus, by “substantially complementary” herein is meant that the probes or primers are sufficiently complementary to the target sequence to hybridize under the selected reaction conditions.

As used herein, the terms “label” refers to detectable moieties that can be attached to an oligonucleotide, mobility probe, or otherwise be used in a reporter system, to thereby render the molecule detectable by an instrument or method. For example, a label can be any moiety that: (i) provides a detectable signal; (ii) interacts with a second label to modify the detectable signal provided by the first or second label; or (iii) confers a capture function, e.g. hydrophobic affinity, antibody/antigen, ionic complexation. The skilled artisan will appreciate that many different species of reporter labels can be used in the present teachings, either individually or in combination with one or more different labels. Exemplary labels include, but are not limited to, fluorophores, radioisotopes, Quantum Dots, chromogens, enzymes, antigens including but not limited to epitope tags, heavy metals, dyes, phosphorescence groups, chemiluminescent groups, electrochemical detection moieties, affinity tags, binding proteins, phosphors, rare earth chelates, near-infrared dyes, including but not limited to, “Cy.7.SPh.NCS,” “Cy.7.OphEt.NCS,” “Cy7.OphEt.CO₂Su”, and IRD800 (see, e.g., J. Flanagan et al., Bioconjug. Chem. 8:751-56 (1997); and DNA Synthesis with IRD800 Phosphoramidite, LI-COR Bulletin #111, LI-COR, Inc., Lincoln, Nebr.), electrochemiluminescence labels, including but not limited to, tris(bipyridal) ruthenium (II), also known as Ru(bpy)₃²⁺, Os(1,10-phenanthroline)₂bis(diphenylphosphino)ethane²⁺, also known as Os(phen)₂(dppene)²⁺, luminol/hydrogen peroxide, Al(hydroxyquinoline-5-sulfonic acid), 9,10-diphenylanthracene-2-sulfonate, and tris(4-vinyl-4′-methyl-2,2′-bipyridal) ruthenium (II), also known as Ru(v-bpy₃²⁺), and the like.

Detailed descriptions of ECL and electrochemiluminescent moieties can be found in, among other places, A. Bard and L. Faulkner, Electrochemical Methods, John Wiley & Sons (2001); M. Collinson and M. Wightman, Anal. Chem. 65:2576 et seq. (1993); D. Brunce and M. Richter, Anal. Chem. 74:3157 et seq. (2002); A. Knight, Trends in Anal. Chem. 18:47 et seq. (1999); B. Muegge et al., Anal. Chem. 75:1102 et seq. (2003); H. Abrunda et al., J. Amer. Chem. Soc. 104:2641 et seq. (1982); K. Maness et al., J. Amer. Chem. Soc. 118:10609 et seq. (1996); M. Collinson and R. Wightman, Science 268:1883 et seq. (1995); and U.S. Pat. No. 6,479,233.

As used herein, the term “fluorophore” refers to a label that comprises a resonance-delocalized system or aromatic ring system that absorbs light at a first wavelength and emits fluorescent light at a second wavelength in response to the absorption event. A wide variety of such dye molecules are known in the art. For example, fluorescent dyes can be selected from any of a variety of classes of fluorescent compounds, such as xanthenes, rhodamines, fluoresceins, cyanines, phthalocyanines, squaraines, and bodipy dyes. In some embodiments, the dye comprises a xanthene-type dye, which contains a fused three-ring system of the form:

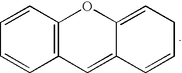

This parent xanthene ring may be unsubstituted (i.e., all substituents are H) or can be substituted with one or more of a variety of the same or different substituents, such as described below. In some embodiments, the dye contains a parent xanthene ring having the general structure:

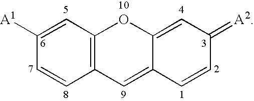

In the parent xanthene ring depicted above, A¹ is OH or NH₂ and A² is O or NH₂⁺. When A¹ is OH and A² is 0, the parent xanthene ring is a fluorescein-type xanthene ring. When A¹ is NH₂ and A² is NH₂⁺, the parent xanthene ring is a rhodamine-type xanthene ring. When A¹ is NH₂ and A² is 0, the parent xanthene ring is a rhodol-type xanthene ring. In the parent xanthene ring depicted above, one or both nitrogens of A¹ and A² (when present) and/or one or more of the carbon atoms at positions C1, C2, C4, C5, C7, C8 and C9 can be independently substituted with a wide variety of the same or different substituents. In some embodiments, typical substituents can include, but are not limited to, —X, —R, —OR, —SR, —NRR, perhalo (C₁-C₆) alkyl, —CX₃, —CF₃, —CN, —OCN, —SCN, —NCO, —NCS, —NO, —NO₂, —N₃, —S(O)₂O⁻, —S(O)₂OH, —S(O)₂R, —C(O)R, —C(O)X, —C(S)R, —C(S)X, —C(O)OR, —C(O)O—, —C(S)OR, —C(O)SR, —C(S)SR, —C(O)NRR, —C(S)NRR and —C(NR)NRR, where each X is independently a halogen (preferably —F or Cl) and each R is independently hydrogen, (C₁-C₆) alkyl, (C₁-C₆) alkanyl, (C₁-C₆) alkenyl, (C₁-C₆) alkynyl, (C₅-C₂₀) aryl, (C₆-C₂₆) arylalkyl, (C₅-C₂₀) arylaryl, heteroaryl, 6-26 membered heteroarylalkyl 5-20 membered heteroaryl-heteroaryl, carboxyl, acetyl, sulfonyl, sulfinyl, sulfone, phosphate, or phosphonate. Moreover, the Cl and C2 substituents and/or the C7 and C8 substituents can be taken together to form substituted or unsubstituted buta[1,3]dieno or (C₅-C₂₀) aryleno bridges. Generally, substituents that do not tend to quench the fluorescence of the parent xanthene ring are preferred, but in some embodiments quenching substituents may be desirable. Substituents that tend to quench fluorescence of parent xanthene rings are electron-withdrawing groups, such as —NO₂, —Br, and —I. In some embodiments, C9 is unsubstituted. In some embodiments, C9 is substituted with a phenyl group. In some embodiments, C9 is substituted with a substituent other than phenyl. When A¹ is NH₂ and/or A² is NH₂⁺, these nitrogens can be included in one or more bridges involving the same nitrogen atom or adjacent carbon atoms, e.g., (C₁-C₁₂) alkyldiyl, (C₁-C₁₂) alkyleno, 2-12 membered heteroalkyldiyl and/or 2-12 membered heteroalkyleno bridges. Any of the substituents on carbons C1, C2, C4, C5, C7, C8, C9 and/or nitrogen atoms at C3 and/or C6 (when present) can be further substituted with one or more of the same or different substituents, which are typically selected from —X, —R′, ═O, —OR′, —SR′, ═S, —NR′R′, ═NR′, —CX₃, —CN, —OCN, —SCN, —NCO, —NCS, —NO, —NO₂, ═N₂, —N₃, —NHOH, —S(O)₂O⁻, —S(O)₂OH, —S(O)₂R′, —P(O)(O—)₂, —P(O)(OH)₂, —C(O)R′, —C(O)X, —C(S)R′, —C(S)X, —C(O)OR′, —C(O)O—, —C(S)OR′, —C(O)SR′, —C(S)SR′, —C(O)NR′R′, —C(S)NR′R′ and —C(NR)NR′R′, where each X is independently a halogen (preferably —F or —Cl) and each R′ is independently hydrogen, (C₁-C₆) alkyl, 2-6 membered heteroalkyl, (C₅-C₁₄) aryl or heteroaryl, carboxyl, acetyl, sulfonyl, sulfinyl, sulfone, phosphate, or phosphonate.

Exemplary parent xanthene rings include, but are not limited to, rhodamine-type parent xanthene rings and fluorescein-type parent xanthene rings.

In one embodiment, the dye contains a rhodamine-type xanthene dye that includes the following ring system:

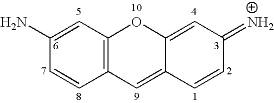

In the rhodamine-type xanthene ring depicted above, one or both nitrogens and/or one or more of the carbons at positions C1, C2, C4, C5, C7 or C8 can be independently substituted with a wide variety of the same or different substituents, as described above for the parent xanthene rings, for example. C9 may be substituted with hydrogen or other substituent, such as an orthocarboxyphenyl or ortho(sulfonic acid)phenyl group. Exemplary rhodamine-type xanthene dyes can include, but are not limited to, the xanthene rings of the rhodamine dyes described in U.S. Pat. Nos. 5,936,087, 5,750,409, 5,366,860, 5,231,191, 5,840,999, 5,847,162, and 6,080,852 (Lee et al.), PCT Publications WO 97/36960 and WO 99/27020, Sauer et al., J. Fluorescence 5(3):247-261 (1995), Arden-Jacob, Neue Lanwellige Xanthen-Farbstoffe für Fluoreszenzsonden und Farbstoff Laser, Verlag Shaker, Germany (1993), and Lee et al., Nucl. Acids Res. 20:2471-2483 (1992). Also included within the definition of “rhodamine-type xanthene ring” are the extended-conjugation xanthene rings of the extended rhodamine dyes described in U.S. application Ser. No. 09/325,243 filed Jun. 3, 1999.

In some embodiments, the dye comprises a fluorescein-type parent xanthene ring having the structure:

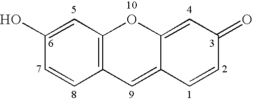

In the fluorescein-type parent xanthene ring depicted above, one or more of the carbons at positions C1, C2, C4, C5, C7, C8 and C9 can be independently substituted with a wide variety of the same or different substituents, as described above for the parent xanthene rings. C9 may be substituted with hydrogen or other substituent, such as an orthocarboxyphenyl or ortho(sulfonic acid)phenyl group. Exemplary fluorescein-type parent xanthene rings include, but are not limited to, the xanthene rings of the fluorescein dyes described in U.S. Pat. Nos. 4,439,356, 4,481,136, 4,933,471 (Lee), U.S. Pat. No. 5,066,580 (Lee), U.S. Pat. Nos. 5,188,934, 5,654,442, and 5,840,999, WO 99/16832, and EP 050684. Also included within the definition of “fluorescein-type parent xanthene ring” are the extended xanthene rings of the fluorescein dyes described in U.S. Pat. Nos. 5,750,409 and 5,066,580. In some embodiments, the dye comprises a rhodamine dye, which can comprise a rhodamine-type xanthene ring in which the C9 carbon atom is substituted with an orthocarboxy phenyl substituent (pendent phenyl group). Such compounds are also referred to herein as orthocarboxyfluoresceins. In some embodiments, a subset of rhodamine dyes are 4,7,-dichlororhodamines. Typical rhodamine dyes can include, but are not limited to, rhodamine B, 5-carboxyrhodamine, rhodamine X (ROX), 4,7-dichlororhodamine X (dROX), rhodamine 6G (R6G), 4,7-dichlororhodamine 6G, rhodamine 110 (R110), 4,7-dichlororhodamine 110 (dR110), tetramethyl rhodamine (TAMRA) and 4,7-dichloro-tetramethylrhodamine (dTAMRA). Additional rhodamine dyes can be found, for example, in U.S. Pat. No. 5,366,860 (Bergot et al.), U.S. Pat. No. 5,847,162 (Lee et al.), U.S. Pat. No. 6,017,712 (Lee et al.), U.S. Pat. No. 6,025,505 (Lee et al.), U.S. Pat. No. 6,080,852 (Lee et al.), U.S. Pat. No. 5,936,087 (Benson et al.), U.S. Pat. No. 6,111,116 (Benson et al.), U.S. Pat. No. 6,051,719 (Benson et al.), U.S. Pat. Nos. 5,750,409, 5,366,860, 5,231,191, 5,840,999, and 5,847,162, U.S. Pat. No. 6,248,884 (Lam et al.), PCT Publications WO 97/36960 and WO 99/27020, Sauer et al., 1995, J. Fluorescence 5(3):247-261, Arden-Jacob, 1993, Neue Lanwellige Xanthen-Farbstoffe für Fluoresenzsonden und Farbstoff Laser, Verlag Shaker, Germany, and Lee et al., Nucl. Acids Res. 20(10):2471-2483 (1992), Lee et al., Nucl. Acids Res. 25:2816-2822 (1997), and Rosenblum et al., Nucl. Acids Res. 25:45004504 (1997), for example. In some embodiments, the dye comprises a 4,7-dichloro-orthocarboxyrhodamine. In some embodiments, the dye comprises a fluorescein dye, which comprises a fluorescein-type xanthene ring in which the C9 carbon atom is substituted with an orthocarboxy phenyl substituent (pendent phenyl group). One typical subset of fluorescein-type dyes are 4,7,-dichlorofluoresceins. Typical fluorescein dyes can include, but are not limited to, 5-carboxyfluorescein (5-FAM), 6-carboxyfluorescein (6-FAM). Additional typical fluorescein dyes can be found, for example, in U.S. Pat. Nos. 5,750,409, 5,066,580, 4,439,356, 4,481,136, 4,933,471 (Lee), U.S. Pat. No. 5,066,580 (Lee), U.S. Pat. No. 5,188,934 (Menchen et al.), U.S. Pat. No. 5,654,442 (Menchen et al.), U.S. Pat. No. 6,008,379 (Benson et al.), and U.S. Pat. No. 5,840,999, PCT publication WO 99/16832, and EPO Publication 050684. In some embodiments, the dye comprises a 4,7-dichloro-orthocarboxyfluorescein. In some embodiments, the dye can be a cyanine, phthalocyanine, squaraine, or bodipy dye, such as described in the following references and references cited therein: U.S. Pat. No. 5,863,727 (Lee et al.), U.S. Pat. No. 5,800,996 (Lee et al.), U.S. Pat. No. 5,945,526 (Lee et al.), U.S. Pat. No. 6,080,868 (Lee et al.), U.S. Pat. No. 5,436,134 (Haugland et al.), U.S. Pat. No. 5,863,753 (Haugland et al.), U.S. Pat. No. 6,005,113 (Wu et al.), and WO 96/04405 (Glazer et al.).

As used herein, the term “identifying portion” refers to a moiety or moieties that can be used to identify a particular probe species and target polynucleotide, and can refer to a variety of distinguishable moieties, including for example labels, zipcodes, mobility modifiers, a known number of nucleobases, and combinations thereof. In some embodiments, identifying portion refers to an oligonucleotide sequence that can be used for separating the element to which it is bound, including without limitation, bulk separation; for tethering or attaching the element to which it is bound to a substrate, which may or may not include separating; for annealing an identifying portion complement that may comprise at least one moiety, such as a mobility modifier, one or more labels, and combinations thereof. In some embodiments, the same identifying portion is used with a multiplicity of different elements to effect: bulk separation, substrate attachment, and combinations thereof. The terms “identifying portion complement” typically refers to at least one oligonucleotide that comprises at least one sequence of nucleobases that are at least substantially complementary to and hybridize with their corresponding identifying portion. In some embodiments, identifying portion complements serve as capture moieties for attaching at least one identifier portion:element complex to at least one substrate; serve as “pull-out” sequences for bulk separation procedures; or both as capture moieties and as pull-out sequences (see for example O'Neil, et al., U.S. Pat. Nos. 6,638,760, 6,514,699, 6,146,511, and 6,124,092). In some embodiments, at least one identifying portion complement comprises at least one reporter group and serves as a label for at least one ligation product, at least one ligation product surrogate, and combinations thereof. In some embodiments, determining comprises detecting one or more reporter groups on at least one identifying portion complement.

Typically, identifying portions and their corresponding identifying portion complements are selected to minimize: internal, self-hybridization; cross-hybridization with different identifying portion species, nucleotide sequences in a reaction composition, including but not limited to gDNA, different species of identifying portion complements, or target-specific portions of probes, and the like; but should be amenable to facile hybridization between the identifying portion and its corresponding identifying portion complement. Identifying portion sequences and identifying portion complement sequences can be selected by any suitable method, for example but not limited to, computer algorithms such as described in PCT Publication Nos. WO 96/12014 and WO 96/41011 and in European Publication No. EP 799,897; and the algorithm and parameters of SantaLucia (Proc. Natl. Acad. Sci. 95:1460-65 (1998)). Descriptions of identifying portions can be found in, among other places, U.S. Pat. No. 6,309,829 (referred to as “tag segment” therein); U.S. Pat. No. 6,451,525 (referred to as “tag segment” therein); U.S. Pat. No. 6,309,829 (referred to as “tag segment” therein); U.S. Pat. No. 5,981,176 (referred to as “grid oligonucleotides” therein); U.S. Pat. No. 5,935,793 (referred to as “identifier tags” therein); and PCT Publication No. WO 01/92579 (referred to as “addressable support-specific sequences” therein).

Identifying portions can be located on at least one end of at least one probe, at least one primer, at least one ligation product, at least one ligation product surrogate, and combinations thereof; or they can be located internally. In some embodiments, at least one identifying portion is attached to at least one probe, at least one primer, at least one ligation product, at least one ligation product surrogate, and combinations thereof, via at least one linker arm. In some embodiments, at least one linker arm is cleavable. In some embodiments, the identifying portion is located on the identifying portion of the first probes.

In some embodiments, identifying portions are at least 12 bases in length, at least 15 bases in length, 12-60 bases in length, or 15-30 bases in length. In some embodiments, at least one identifying portion is 12, 15, 20, 21, 22, 23, 24, 25, 26, 27, 28, 29, 30, 45, or 60 bases in length. In some embodiments, at least two identifying portion: identifying portion complement duplexes have melting temperatures that fall within a Δ Tₘ range (Tₘₐₓ−Tₘᵢₙ) of no more than 10° C. of each other. In some embodiments, at least two identifying portion: identifying portion complement duplexes have melting temperatures that fall within a Δ Tₘ range of 5° C. or less of each other. In some embodiments, at least two identifying portion: identifying portion complement duplexes have melting temperatures that fall within a Δ Tₘ range of 2° C. or less of each other.

In some embodiments, at least one identifying portion or at least one identifying portion complement is used to separate the element to which it is bound from at least one component of a ligation reaction composition, a digestion reaction composition, an amplified ligation reaction composition, or the like. In some embodiments, identifying portions are used to attach at least one ligation product, at least one ligation product surrogate, or combinations thereof, to at least one substrate. In some embodiments, at least one ligation product, at least one ligation product surrogate, or combinations thereof, comprise the same identifying portion. Examples of separation approaches include but are not limited to, separating a multiplicity of different element: identifying portion species using the same identifying portion complement, tethering a multiplicity of different element: identifying portion species to a substrate comprising the same identifying portion complement, or both. In some embodiments, at least one identifying portion complement comprises at least one label, at least one mobility modifier, at least one label binding portion, or combinations thereof. In some embodiments, at least one identifying portion complement is annealed to at least one corresponding identifying portion and, subsequently, at least part of that identifying portion complement is released and detected.

The term “mobility modifier” as used herein refers to at least one molecular entity, for example but not limited to, at least one polymer chain, that when added to at least one element (e.g., at least one probe, at least one primer, at least one ligation product, at least one ligation product surrogate, at least one mobility probe, or combinations thereof) affects the mobility of the element to which it is hybridized or bound, covalently or non-covalently, in at least one mobility-dependent analytical technique. Typically, a mobility modifier changes the charge/translational frictional drag when hybridized or bound to the element; or imparts a distinctive mobility, for example but not limited to, a distinctive elution characteristic in a chromatographic separation medium or a distinctive electrophoretic mobility in a sieving matrix or non-sieving matrix, when hybridized or bound to the corresponding element; or both (see, e.g., U.S. Pat. Nos. 5,470,705 and 5,514,543). In some embodiments, a multiplicity of probes exclusive of mobility modifiers, a multiplicity of primers exclusive of mobility modifiers, a multiplicity of ligation products exclusive of mobility modifiers, a multiplicity of ligation product surrogates exclusive of mobility modifiers, or combinations thereof, have the same or substantially the same mobility in at least one mobility-dependent analytical technique. For various examples of mobilitity modifiers see for example U.S. Pat. Nos. 6,395,486, 6,358,385, 6,355,709, 5,916,426, 5,807,682, 5,777,096, 5,703,222, 5,556,7292, 5,567,292, 5,552,028, 5,470,705, and Barbier et al., Current Opinion in Biotechnology, 2003, 14:1:51-57

In some embodiments, a multiplicity of probes, a multiplicity of primers, a multiplicity of ligation products, a multiplicity of ligation product surrogates, or combinations thereof, have substantially similar distinctive mobilities, for example but not limited to, when a multiplicity of elements comprising mobility modifiers have substantially similar distinctive mobilities so they can be bulk separated or they can be separated from other elements comprising mobility modifiers with different distinctive mobilities. In some embodiments, a multiplicity of probes comprising mobility modifiers, a multiplicity of primers comprising mobility modifiers, a multiplicity of ligation products comprising mobility modifiers, a multiplicity of ligation product surrogates comprising mobility modifiers, at least one mobility probe, or combinations thereof, have different distinctive mobilities.

In some embodiments, at least one mobility modifier comprises at least one nucleotide polymer chain, including without limitation, at least one oligonucleotide polymer chain, at least one polynucleotide polymer chain, or both at least one oligonucleotide polymer chain and at least one polynucleotide polymer chain (see for example Published P.C.T. application WO9615271A1, as well as product literature for Keygene SNPWave™ for some examples of using known numbers of nucleotides to confer mobility to ligation products). In some embodiments, at least one mobility modifier comprises at least one non-nucleotide polymer chain. Exemplary non-nucleotide polymer chains include, without limitation, peptides, polypeptides, polyethylene oxide (PEO), or the like. In some embodiments, at least one polymer chain comprises at least one substantially uncharged, water-soluble chain, such as a chain composed of PEO units; a polypeptide chain; or combinations thereof.

The polymer chain can comprise a homopolymer, a random copolymer, a block copolymer, or combinations thereof. Furthermore, the polymer chain can have a linear architecture, a comb architecture, a branched architecture, a dendritic architecture (e.g., polymers containing polyamidoamine branched polymers, Polysciences, Inc. Warrington, Pa.), or combinations thereof. In some embodiments, at least one polymer chain is hydrophilic, or at least sufficiently hydrophilic when hybridized or bound to an element to ensure that the element-mobility modifier is readily soluble in aqueous medium. Where the mobility-dependent analysis technique is electrophoresis, in some embodiments, the polymer chains are uncharged or have a charge/subunit density that is substantially less than that of its corresponding element.

The synthesis of polymer chains useful as mobility modifiers will depend, at least in part, on the nature of the polymer. Methods for preparing suitable polymers generally follow well-known polymer subunit synthesis methods. These methods, which involve coupling of defined-size, multi-subunit polymer units to one another, either directly or through charged or uncharged linking groups, are generally applicable to a wide variety of polymers, such as polyethylene oxide, polyglycolic acid, polylactic acid, polyurethane polymers, polypeptides, oligosaccharides, and nucleotide polymers. Such methods of polymer unit coupling are also suitable for synthesizing selected-length copolymers, e.g., copolymers of polyethylene oxide units alternating with polypropylene units. Polypeptides of selected lengths and amino acid composition, either homopolymer or mixed polymer, can be synthesized by standard solid-phase methods (e.g., Int. J. Peptide Protein Res., 35: 161-214 (1990)).

One method for preparing PEO polymer chains having a selected number of hexaethylene oxide (HEO) units, an HEO unit is protected at one end with dimethoxytrityl (DMT), and activated at its other end with methane sulfonate. The activated HEO is then reacted with a second DMT-protected HEO group to form a DMT-protected HEO dimer. This unit-addition is then carried out successively until a desired PEO chain length is achieved (e.g., U.S. Pat. No. 4,914,210; see also, U.S. Pat. No. 5,777,096).

As used herein, a “mobility probe” generally refers to a molecule comprising a mobility modifier, a label, and an identifying portion or identifying portion complement that can hybridize to a ligation product or ligation product surrogate, the detection of which allows for the identification of the target polynucleotide.

As used herein, the term “mobility-dependent analytical technique” as used herein refers to any means for separating different molecular species based on differential rates of migration of those different molecular species in one or more separation techniques. Exemplary mobility-dependent analysis techniques include gel electrophoresis, capillary electrophoresis, chromatography, capillary electrochromatography, mass spectroscopy, sedimentation, e.g., gradient centrifugation, field-flow fractionation, multi-stage extraction techniques and the like. Descriptions of mobility-dependent analytical techniques can be found in, among other places, U.S. Pat. Nos. 5,470,705, 5,514,543, 5,580,732, 5,624,800, and 5,807,682, PCT Publication No. WO 01/92579, Fu et al., Current Opinion in Biotechnology, 2003, 14:1:96-100, D. R. Baker, Capillary Electrophoresis, Wiley-Interscience (1995), Biochromatography: Theory and Practice, M. A. Vijayalakshmi, ed., Taylor & Francis, London, U.K. (2003); and A. Pingoud et al., Biochemical Methods: A Concise Guide for Students and Researchers, Wiley-VCH Verlag GmbH, Weinheim, Germany (2002).

As used herein, the term “ligation agent”, according to the present invention, can comprise any number of enzymatic or non-enzymatic reagents. For example, ligase is an enzymatic ligation reagent that, under appropriate conditions, forms phosphodiester bonds between the 3′-OH and the 5′-phosphate of adjacent nucleotides in DNA molecules, RNA molecules, or hybrids. Temperature sensitive ligases, include, but are not limited to, bacteriophage T4 ligase and E. coli ligase. Thermostable ligases include, but are not limited to, Afu ligase, Taq ligase, Tfl ligase, Tth ligase, Tth HB8 ligase, Thermus species AK16D ligase and Pfu ligase (see for example Published P.C.T. Application WO00/26381, Wu et al., Gene, 76(2):245-254, (1989), Luo et al., Nucleic Acids Research, 24(15): 3071-3078 (1996). The skilled artisan will appreciate that any number of thermostable ligases, including DNA ligases and RNA ligases, can be obtained from thermophilic or hyperthermophilic organisms, for example, certain species of eubacteria and archaea; and that such ligases can be employed in the disclosed methods and kits. Further, reversibly inactivated enzymes (see for example U.S. Pat. No. 5,773,258) can be employed in some embodiments of the present teachings.

Chemical ligation agents include, without limitation, activating, condensing, and reducing agents, such as carbodiimide, cyanogen bromide (BrCN), N-cyanoimidazole, imidazole, 1-methylimidazole/carbodiimide/cystamine, dithiothreitol (DTT) and ultraviolet light. Autoligation, i.e., spontaneous ligation in the absence of a ligating agent, is also within the scope of the teachings herein. Detailed protocols for chemical ligation methods and descriptions of appropriate reactive groups can be found in, among other places, Xu et al., Nucleic Acid Res., 27:875-81 (1999); Gryaznov and Letsinger, Nucleic Acid Res. 21:1403-08 (1993); Gryaznov et al., Nucleic Acid Res. 22:2366-69 (1994); Kanaya and Yanagawa, Biochemistry 25:7423-30 (1986); Luebke and Dervan, Nucleic Acids Res. 20:3005-09 (1992); Sievers and von Kiedrowski, Nature 369:221-24 (1994); Liu and Taylor, Nucleic Acids Res. 26:3300-04 (1999); Wang and Kool, Nucleic Acids Res. 22:2326-33 (1994); Purmal et al., Nucleic Acids Res. 20:3713-19 (1992); Ashley and Kushlan, Biochemistry 30:2927-33 (1991); Chu and Orgel, Nucleic Acids Res. 16:3671-91 (1988); Sokolova et al., FEBS Letters 232:153-55 (1988); Naylor and Gilham, Biochemistry 5:2722-28 (1966); and U.S. Pat. No. 5,476,930.

Photoligation using light of an appropriate wavelength as a ligation agent is also within the scope of the teachings. In some embodiments, photoligation comprises probes comprising nucleotide analogs, including but not limited to, 4-thiothymidine (s⁴T), 5-vinyluracil and its derivatives, or combinations thereof. In some embodiments, the ligation agent comprises: (a) light in the UV-A range (about 320 nm to about 400 nm), the UV-B range (about 290 nm to about 320 nm), or combinations thereof, (b) light with a wavelength between about 300 nm and about 375 nm, (c) light with a wavelength of about 360 nm to about 370 nm; (d) light with a wavelength of about 364 nm to about 368 nm, or (e) light with a wavelength of about 366 nm. In some embodiments, photoligation is reversible. Descriptions of photoligation can be found in, among other places, Fujimoto et al., Nucl. Acid Symp. Ser. 42:3940 (1999); Fujimoto et al., Nucl. Acid Res. Suppl. 1:185-86 (2001); Fujimoto et al., Nucl. Acid Suppl., 2:155-56 (2002); Liu and Taylor, Nucl. Acid Res. 26:3300-04 (1998) and on the world wide web at: sbchem.kyoto-u.ac.jp/saito-lab.

Ligation

Ligation according to the present teachings comprises any enzymatic or non-enzymatic process wherein an inter-nucleotide linkage is formed between the opposing ends of nucleic acid sequences that are adjacently hybridized to a template. Typically, the opposing ends of the annealed nucleic acid probes are suitable for ligation (suitability for ligation is a function of the ligation means employed). In some embodiments, ligation also comprises at least one gap-filling procedure, wherein the ends of the two probes are adjacent but not contiguoulsy hybridized initially, but the 3′-end of the first probe is extended by one or more nucleotide until it is contiguous to the 5′-end of the second probe, typically by a polymerase (see, e.g., U.S. Pat. No. 6,004,826). The internucleotide linkage can include, but is not limited to, phosphodiester bond formation. Such bond formation can include, without limitation, those created enzymatically by at least one DNA ligase or at least one RNA ligase, for example but not limited to, T4 DNA ligase, T4 RNA ligase, Thermus thermophilus (Tth) ligase, Thermus aquaticus (Taq) DNA ligase, Thermus scotoductus (Tsc) ligase, TS2126 (a thermophilic phage that infects Tsc) RNA ligase, Archaeoglobus flugidus (Afu) ligase, Pyrococcus furiosus (Pfu) ligase, or the like, including but not limited to reversibly inactivated ligases (see, e.g., U.S. Pat. No. 5,773,258), and enzymatically active mutants and variants thereof.

Other internucleotide linkages include, without limitation, covalent bond formation between appropriate reactive groups such as between an α-haloacyl group and a phosphothioate group to form a thiophosphorylacetylamino group, a phosphorothioate a tosylate or iodide group to form a 5′-phosphorothioester, and pyrophosphate linkages.

Chemical ligation can, under appropriate conditions, occur spontaneously such as by autoligation. Alternatively, “activating” or reducing agents can be used. Examples of activating and reducing agents include, without limitation, carbodiimide, cyanogen bromide (BrCN), imidazole, 1-methylimidazole/carbodiimide/cystamine, N-cyanoimidazole, dithiothreitol (DTT) and ultraviolet light, such as used for photoligation.

Ligation generally comprises at least one cycle of ligation, i.e., the sequential procedures of: hybridizing the target-specific portions of a first probe and a corresponding second probe to their respective complementary regions on the corresponding target nucleic acid sequences; ligating the 3′ end of the first probe with the 5′ end of the second probe to form a ligation product; and denaturing the nucleic acid duplex to release the ligation product from the ligation product:target nucleic acid sequence duplex. The ligation cycle may or may not be repeated, for example, without limitation, by thermocycling the ligation reaction to amplify the ligation product using ligation probes (as distinct from using primers and polymerase to generate amplified ligation products).

Also within the scope of the teachings are ligation techniques such as gap-filling ligation, including, without limitation, gap-filling versions OLA, LDR, LCR, FEN-cleavage mediated versions of OLA, LDR, and LCR, bridging oligonucleotide ligation, correction ligation, and looped linker-based concatameric ligation. Additional non-limiting ligation techniques included within the present teachings comprise OLA followed by PCR (see for example Rosemblum et al, P.C.T. Application US03/37227, Rosemblum et al., P.C.T. Application US03/37212 and Barany et al., Published P.C.T. application WO974559A1, OLA comprising mobility modifiers (see for example U.S. Pat. No. 5,514,543, PCR followed by OLA, two PCR's followed by an OLA, ligation comprising single circularizable probes (see for example Landregren et al., WO9741254A1, OLA comprising rolling circle replication of padlock probes (see for example Landregren et al., U.S. Pat. No. 6,558,928. Additional descriptions of these and related techniques can be found in, among other places, U.S. Pat. Nos. 5,185,243 and 6,004,826, 5,830,711, 6,511,810, 6,027,889; published European Patent Applications EP 320308 and EP 439182; Published PCT applications WO 90/01069, WO 01/57268, WO0056927A3, WO9803673A1, WO200117329, Landegren et al., Science 241:1077-80 (1988), Day et al., Genomics, 29(1): 152-162 (1995), de Arruda et al., and U.S. Application 60/517,470. In some embodiments ligation can provide for sample preparation prior to a subsequent amplification step. In some embodiments ligation can provide amplification in and of itself, as well as provide for an initial amplification followed by a subsequent amplification.

In some embodiments of the present teachings, unconventional nucleotide bases can be introduced into the ligation probes and the resulting products treated by enzymatic (e.g., glycosylases) and/or physical-chemical means in order to render the product incapable of acting as a template for subsequent subsequent reactions such as amplification. In some embodiments, uracil can be included as a nucleobase in the ligation reaction mixture, thereby allowing for subsequent reactions to decontaminate carryover of previous uracil-containing products by the use of uracil-N-glycosylase. Various approaches to decontamination using glycosylases and the like can be found for example in Published P.C.T. Application WO9201814A2.

Methods for removing unhybridized and/or unligated probes following a ligation reaction are known in the art, and are further discussed infra. Such procedures include nuclease-mediated approaches, dilution, size exclusion approaches, affinity moiety procedures, (see for example U.S. Provisional Application 60/517,470, U.S. Provisional Application 60/477,614, and P.C.T. Application 2003/37227), affinity-moiety procedures involving immobilization of target polynucleotides (see for example Published P.C.T. Application WO 03/006677A2).

Amplification

Amplification according to the present teachings encompass any manner by which at least a part of at least one target polynucleotide, ligation product, at least one ligation product surrogate, or combinations thereof, is reproduced, typically in a template-dependent manner, including without limitation, a broad range of techniques for amplifying nucleic acid sequences, either linearly or exponentially. Exemplary steps for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA) and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction—CCR), and the like. Descriptions of such techniques can be found in, among other places, Sambrook and Russell; Sambrook et al.; Ausbel et al.; PCR Primer: A Laboratory Manual, Diffenbach, Ed., Cold Spring Harbor Press (1995); The Electronic Protocol Book, Chang Bioscience (2002)(“The Electronic Protocol Book”); Msuih et al., J. Clin. Micro. 34:501-07 (1996); The Nucleic Acid Protocols Handbook, R. Rapley, ed., Humana Press, Totowa, N.J. (2002)(“Rapley”); Abramson et al., Curr Opin Biotechnol. 1993 February; 4(1):41-7, U.S. Pat. No. 6,027,998; U.S. Pat. No. 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18- (2002); Lage et al., Genome Res. 2003 February; 13(2):294-307, and Landegren et al., Science 241:1077-80 (1988), Demidov, V., Expert Rev Mol Diagn. 2002 November; 2(6):542-8., Cook et al., J Microbiol Methods. 2003 May;53(2):165-74, Schweitzer et al., Curr Opin Biotechnol. 2001 February; 12(1):21-7, U.S. Pat. No. 5,830,711, U.S. Pat. No. 6,027,889, U.S. Pat. No. 5,686,243, Published P.C.T. Application WO0056927A3, and Published P.C.T. Application WO9803673A1.

In some embodiments, amplification comprises at least one cycle of the sequential procedures of: hybridizing at least one primer with complementary or substantially complementary sequences in at least one ligation product, at least one ligation product surrogate, or combinations thereof; synthesizing at least one strand of nucleotides in a template-dependent manner using a polymerase; and denaturing the newly-formed nucleic acid duplex to separate the strands. The cycle may or may not be repeated. Amplification can comprise thermocycling or can be performed isothermally. In some embodiments, newly-formed nucleic acid duplexes are not initially denatured, but are used in their double-stranded form in one or more subsequent steps.

Primer extension is an amplifying step that comprises elongating at least one probe or at least one primer that is annealed to a template in the 5′ to 3′ direction using an amplifying means such as a polymerase. According to some embodiments, with appropriate buffers, salts, pH, temperature, and nucleotide triphosphates, including analogs thereof, i.e., under appropriate conditions, a polymerase incorporates nucleotides complementary to the template strand starting at the 3′-end of an annealed probe or primer, to generate a complementary strand. In some embodiments, primer extension can be used to fill a gap between two probes of a probe set that are hybridized to target sequences of at least one target nucleic acid sequence so that the two probes can be ligated together. In some embodiments, the polymerase used for primer extension lacks or substantially lacks 5′ exonuclease activity.

In some embodiments of the present teachings, unconventional nucleotide bases can be introduced into the amplification reaction products and the products treated by enzymatic (e.g., glycosylases) and/or physical-chemical means in order to render the product incapable of acting as a template for subsequent amplifications. In some embodiments, uracil can be included as a nucleobase in the reaction mixture, thereby allowing for subsequent reactions to decontaminate carrover of previous uracil-containing products by the use of uracil-N-glycosylase (see for example Published P.C.T. Application WO9201814A2). In some embodiments of the present teachings, any of a variety of techniques can be employed prior to amplification in order to facilitate amplification success, as described for example in Radstrom et al., Mol Biotechnol. 2004 February; 26(2):133-46. In some embodiments, amplification can be achieved in a self-contained integrated approach comprising sample preparation and detection, as described for example in U.S. Pat. Nos. 6,153,425 and 6,649,378.

Removal of Unincorporated and/or Undesired Reaction Components

It will be appreciated that reactions involving complex mixtures of nucleic acids in which a number of reactive steps are employed can result in a variety of unincorporated reactions components, and that removal of such unincorporated reaction components by any of a variety of complexity reduction procedures can improve the efficiency and specificity or subsequently occurring reactions.

In some embodiments, complexity reduction includes selective immobilization of target nucleic acids. For example, target nucleic acids can be preferentially immobilized on a solid support. In some embodiments, photo-biotin can be attached to target nucleic acids, and the resulting biotin-labeled nucleic acids immobilized on a solid support comprising an affinity-moiety binder such as streptavidin. Immobilized target nucleic acids can be queried with probes, and non-hybidized and/or non-ligated probes removed by washing (See for Example Published P.C.T. Application WO 03/006677 and U.S. Ser. No. 09/931,285, for further elaboration on such complexity reduction approaches). A variety of washing conditions can be employed, as described for example in recent editions of Ausubel et al., and Maniatis et al.,

In some embodiments, unincorporated probes can be removed by a variety of enzymatic means, wherein for example unprotected 3′ probe ends can be digested with 3′-acting nucleases, 5′ phosphate-bearing probes ends can be digested with 5′-acting nucleases. In some embodiments, such nuclease-digestion mediated approaches to removal of unincorporated reaction components such as ligation probes can further comprise the use of looped-linker probes and/or linkers lacking loops, as described for example in U.S. application 60/517,470, also see infra.

In some embodiments, unreacted ligation probes can be removed from the mixture whereby the first probe can comprise a label and the second probe can be blocked at its 3′ end with an exonuclease blocking moiety. After ligation and the introduction of the nuclease, the labeled unligated first probe can be digested, leaving the ligation product and the second probe. However, since the second probe is unlabelled, it is effectively silent in the assay. In some embodiments, the target polynucleotides are immobilized, and the ligation product can be eluted and detected. In some embodiments, the 3′ end of the second probe further comprises an affinity moiety, and the ligation products and unincorporated second probes can be immobilized with an affinity-moiety binder. In some embodiments, mobility probes can be hybridized to the immobilized ligation products, unhybridized mobility probes washed away, and hybridized mobility probes eluted and detected. In some embodiments the 5′ endo of the first probe comprises an affinity moiety, and the ligation products and unincorporated first probes can be immobilized with an affinity-moiety binder.

In some embodiments, products from previous reactions performed for example in the same laboratory workspace can contaminate a reaction of interest. In some embodiments, uracil can be incorporated into for example a PCR amplification step, thereby rendering reaction products comprising uracil instead of, or along with, thymidine. In some embodiments, uracil-N-glycosylase can be included in the OLA reaction mixture is such fashion as to degrade uracil-containing contaminants. In some embodiments, a uracil-N-glycosylase mediated clean-up procedure can be implemented in the context of a ligation mixture.

Detection and Quantification

Detection and quantification can be carried out using a variety of procedures, including for example mobility dependent analysis techniques (for example capillary or gel electrophoresis), solid support comprising array capture oligonucleotides, various bead approaches (see for example Published P.C.T. Application WO US02/37499), including fiber optics, as well as flow cytometry (for example, FACS).

The use of capillary and gel electrophoresis for detection and quantification of target polynucleotides is well known, see for example, Grossman, et. al., “High-density Multiplex Detection of Nucleic Acid Sequences: Oligonucleotide Ligation Assay and Sequence-coded Separation,” Nucl. Acids Res. 22(21): 4527-34 (1994), Slater et al., Current Opinion in Biotechnology, 2003, 14:1:58-64, product literature for the Applied Biosystems 3100, 3700, and 3730 capillary electrophoresis instruments, and product literature for the SNPlex Genotyping System Chemistry Guide, also from Applied Biosystems.

Additional mobility dependent analysis techniques that can provide for detection and quantification according to the present teachings include mass spectroscopy (optionally comprising a deconvolution step via chromatography), collision-induced dissociation (CID) fragmentation analysis, fast atomic bombardment and plasma desorption, and electrospray/ionspray (ES) and matrix-assisted laser deorption/ionization (MALDI) mass spectrometry. In some embodiments, MALDI mass spectrometry can be used with a time-of-flight (TOF) configuration (MALDI-TOF, see for example Published P.C.T. Application WO 97/33000), and MALDI-TOF-TOF (see for example Applied Biosystems 4700 Proteomics Discovery System product literature). Additional mass spectrometry approaches for detection and quantification are described for example in the Applied Biosystems Qtrap LC/MS/MS System product literature, the Applied Biosystems QSTAR XL Hybrid LC/MS/MS System product literature, the Applied Biosystems Q TRAP™ LC/MS/MS System product literature, and the Applied Biosystems Voyager-DE™ PRO Biospectrometry Workstation product literature.

The use of a solid support with an array of capture oligonucleotides is fully disclosed among other places in pending provisional U.S. patent application Ser. No. 60/011,359. In some embodiments when using such arrays, the oligonucleotide primers or probes used in the herein-described PCR and/or LDR phases, respectively, can have an addressable hybridization tag (for example, an identifying portion). After the LDR or PCR phases are completed, the addressable hybridization tags of the products of such processes remain single stranded and are caused to hybridize to the capture oligonucleotides during a capture phase. See for example, C. Newton, et al., “The Production of PCR Products With 5′ Single-Stranded Tails Using Primers That Incorporate Novel Phosphoramidite Intermediates,” Nucl. Acids Res. 21(5):1155-62 (1993), Carrino Published P.C.T. Application WO 096152371A1. The present teachings further contemplate a variety of additional array-based procedures known in the art, including but not limited to dot-blots (see for example Andersen and Young, in Nucleic Acid Hybridization-A Practical Approach, IRL Press, Chapter 4, pp. 73-111, 1985, and EPA 0228075, and for the detection of overlapping dines and the construction of genomic maps Evans, G. A. U.S. Pat. No. 5,219,726), reverse dot blots, and matrix hybridization (see Beattie et al., in The 1992 San Diego Conference: Genetic Recognition, November, 1992), photolithographically generated arrays (see for example Fodor et al., 1991, Science, 251: 767-777. as well as Geneflex Tag Arrays from Affymetrix), universal arrays as described for example in Published P.C.T. application WO 9731256A2, WO 0179548A2, WO 0056927A3, product literature associated with commercially available spotted arrays from Agilent, product literature associated with the commercially available Applied Biosystems Expression Array System, printing-based arrays commercially available from Hewlett Packard and Rosetta-Merck, electrode arrays, three dimensional “gel pad” arrays, as well as three-dimensional array methods such as FACS. In some embodiments, detection and quantification can be carried out on a variety of bead-based formats, described for example in Published P.C.T. Applications US98/21193, US99/14387, US98/05025, WO 98/50782, U.S. Ser. Nos. 09/287,573, 09/151,877, 09/256,943, 09/316,154, 60/119,323, and 09/315,584. Also see “Microsphere Detection Guide” from Gangs Laboratories, Fishers Ind. for a discussion of beads and microspheres. In some embodiments, detection and quantification can be carried out with a fiber bundle or array, as is generally described in U.S. Ser. Nos. 08/944,850 and 08/519,062, PCT US 98/05025, and PCT US 98/09163, as well as U.S. Ser. No. 09/473,904.

In some embodiments, during the capture phase of the process the mixture can be contacted with the solid support at an appropriate temperature and for a time period of up to 60 minutes. In some embodiments, during the capture phase of the process the mixture can be contacted with the solid support for an overnight period, or longer. Hybridizations can be accelerated by adding cations, volume exclusion compounds or chaotropic agents. When an array consists of dozens to hundreds of addresses, the correct ligation product sequences can have an opportunity to hybridize to the appropriate address. This may be achieved by the thermal motion of oligonucleotides at the high temperatures used, by mechanical movement of the fluid in contact with the array surface, or by moving the oligonucleotides across the array by electric fields. After hybridization, the array can be washed sequentially with a low stringency wash buffer and then a high stringency wash buffer.

In some embodiments capture oligonucleotides and addressable nucleotide sequences are chosen that will hybridize in a stable fashion. This can involve oligonucleotide sets and the capture oligonucleotides that are configured so that the oligonucleotide sets hybridize to the target nucleotide sequences at a temperature less than that which the capture oligonucleotides hybridize to the addressable hybridization tag. Unless the oligonucleotides are designed in this fashion, false positive signals can result due to capture of adjacent unreacted oligonucleotides from the same oligonucleotide set which are hybridized to the target.

The capture oligonucleotides can be in the form of ribonucleotides, deoxyribonucleotides, modified ribonucleotides, modified deoxyribonucleotides, peptide nucleotide analogues, modified peptide nucleotide analogues, modified phosphate-sugar backbone oligonucleotides, nucleotide analogues, and mixtures thereof.

Where an array is utilized, the detection phase of the process involves scanning and identifying if OLA, LDR, and/or PCR products and the like have been produced and correlating the presence of such products to a presence or absence of the target nucleotide sequence in the test sample. Scanning can be carried out by scanning electron microscopy, confocal microscopy, charge-coupled device, scanning tunneling electron microscopy, infrared microscopy, atomic force microscopy, electrical conductance, and fluorescent or phosphor imaging. Correlating is carried out with a computer.

The present teachings further contemplate the use of various nano-technological-based approaches, as described for example in Alivisatos, A. P. 2002, Scientific American, Inc. in Understanding Nanotechnology, “Less is More in Medicine”, including for example various magnetic tags, gold particles, cantilevers, Quantum Dots, and microfluidic-based approaches (also see for example Schultz et al., Current Opinion in Biotechnology, 2003, 14:1:13-22, Obata et al., Pharmacogenomics. 2002 September; 3(5):697-708, Paegel et al., Curr Opin Biotechnol. 2003 February; 14(1):42-50, U.S. Pat. Nos. 6,670,153, 6,648,015, 6,632,655, 6,620,625, 6,613,581, as well as commercially available products generally available from Caliper and Fluidigm.

In some embodiments of the present teachings, analysis of detected products can be undertaken with the application of various software procedures. For example, analysis of capillary electrophoresis products can employ various commercially available software packages from Applied Biosystems, for example GeneMapper version 3.5 and BioTrekker version 1.0.

In general, it will be appreciated that the process employed for detection and quantification is not a limitation of the present teachings.

Aspects of the present teachings can be further understood in light of the following examples, which should not be construed as limiting the scope of the teachings in anyway.

### Exemplary Embodiments

Numerous fields in molecular biology require the detection of target polynucleotide sequences. The increasing amount of sequence information available to scientists in the post-genomics era has produced an increased need for rapid, reliable, low-cost, high-throughput, sensitive, and accurate methods to query complex nucleic acid samples. Hybridization, ligation, and amplification are procedures frequently employed to detect target polynucleotides (for example, see Cao, Trends in Biotechnology, 22:1:38-44, for a recent review). The reaction mixture complexity, and multiplicity of steps of such procedures produces complex data sets, wherein the degree of false positive rates, false negative rates, and other parameters relevant to assessing reaction integrity, can be difficult to determine in the absence of extensive reaction controls.

Ligation assays are one example in which it can be difficult to interpret a negative result. For example, a negative result in a ligation assay can be an indication of the absence of a particular target polynucleotide sequence (for example, the absence of a particular allelic variant) in the reaction mixture. However, a negative result in a ligation assay can also be an indication of nonfunctional reaction components. The experimentalist cannot necessarily correctly infer that a negative result for particular target polynucleotide sequence in fact represents that the particular target polynucleotide sequence is absent from the reaction mixture when the reaction comprises nonfunctional reaction components.

Some embodiments of the present teachings provide control compositions, kits, and methods for detecting a non-specific ligation product. Some embodiments of the present teachings provide negative control probes in a ligation assay. Such negative control probes can hybridize to monomorphic target polynucleotides with their target specific portions, but fail to hybridize in their discriminating regions, and can provide the experimentalist with a measure of the extent non-specific ligation occurs. The presence of a signal from negative control probes can provide the experimentalist with a measure of non-specific ligation. Such a measure of non-specific ligation can be used to assess the likelihood and/or degree that non-specific ligation and other undesired effects are contaminating assay results.

It can also be difficult to interpret a positive result in a ligation assay. For example, a positive result in a ligation assay can be an indication of the presence of a particular target polynucleotide sequence in the reaction mixture. However, a positive result in a ligation assay can also be an indication of non-specific interactions between reaction components. For example, a positive result can be an indication of non-specific ligation between reaction components, as well as an indication of contamination due to amplifiable polynucleotide sequences from previous reactions. The experimentalist cannot necessarily correctly infer that a positive result for a particular target polynucleotide sequence in fact represents that the particular target polynucleotide sequence is present in the reaction mixture given the possibility of such non-specific interactions occurring in the reaction.

Some embodiments of the present teachings provide control compositions, kits, and methods for detecting a specific ligation product. Some embodiments of the present teachings provide positive control probes in a ligation assay. Such positive control probes can hybridize to monomorphic target polynucleotides, and can provide the experimentalist with verification that the necessary reaction components are functioning in such a way as to provide a positive signal. The presence of such a positive signal from positive control probes can allow the experimentalist to rule out other variables as the cause of a negative result. Such a measure of specific ligation can be used to assess the likelihood and/or degree that non-specific ligation and other undesired effects are contaminating assay results.

The difficulties in assessing specific and non-specific ligation in an olignoucleotide ligation assay can be exacerbated as reaction complexity increases. For example, various approaches of pairing ligation assays with other techniques (see for example Cao et al., 2004), as well as the increasing desire to perform highly multiplexed reactions querying a plurality of target polynucleotide sequences, has resulted in increased reaction complexity, and a resulting increased recognition of the importance of accurate methodologies for determining reaction accuracy.

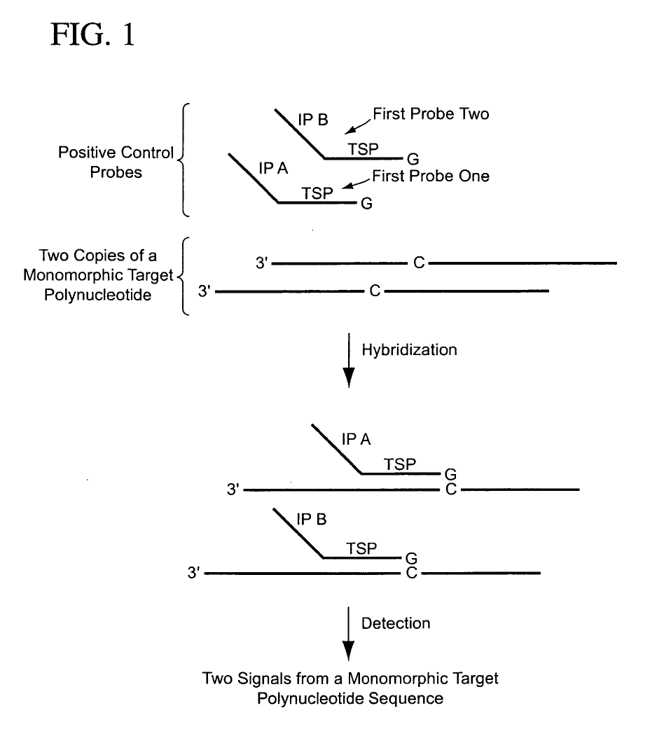

It will be appreciated that while in some embodiments of the present teachings control probes are used in the context of a ligation assay, the present teachings also can more broadly pertain to the ability to generate more than one signal from a target polynucleotide sequence, and need not necessarily involve a ligation assay. In one non-limiting embodiment depicted in FIG. 1, a first positive control probe one and a first positive control probe two can each comprise identical target specific portions (TSP) and disciminating regions (here, a G) that can hybridize to a monomorphic target polynucleotide sequence. The first positive control probe one and the first positive control probe two can further comprise distinct identifying portions (here, IP A for first positive control probe one and IP B for first positive control probe two). Subsequent to hybridization of the first positive control probes to the monomorphic target polynucleotide sequence, one or more steps can be performed to separate those positive control probes that hybridized to the monomorphic target polynucleotide sequence from those probes that did not hybridize to the monomorphic target polynucleotide sequence. Detection of positive control first probe one and positive control first probe two that hybridized to the monomorphic target polynucleotide sequence can result in the production of two distinct signals from a monomorphic target polynucleotide.

In some embodiments of the present teachings positive control probes are used in the context of a ligation assay to generate more than one signal from a target polynucleotide sequence. In one non-limiting embodiment depicted in FIG. 2, a first positive control probe one and a first positive control probe two can each comprise identical target specific portions (TSP) and disciminating nucleotides (here, an A) that can hybridize to a monomorphic target polynucleotide sequence X. A second positive control probe can also hybridize to monomorphic target polynucleotide sequence X. The first positive control probe one and the first positive control probe two can further comprise distinct identifying portions (here, IP A for first positive control probe one and IP B for first positive control probe two). Following hybridization of the first positive control probes and the second positive control probe to adjacent regions on the monomorphic target polynucleotide sequence X, a ligation agent can ligate the first probes to the second probes. One or more steps can be performed to separate those first probes and second probes that hybridized and were ligated from those first probes and second probes that did not hybridize and/or did not ligate. Detection of the resulting ligation products, or ligation product surrogates, can result in the production of two distinct signals from a monomorphic target polynucleotide.

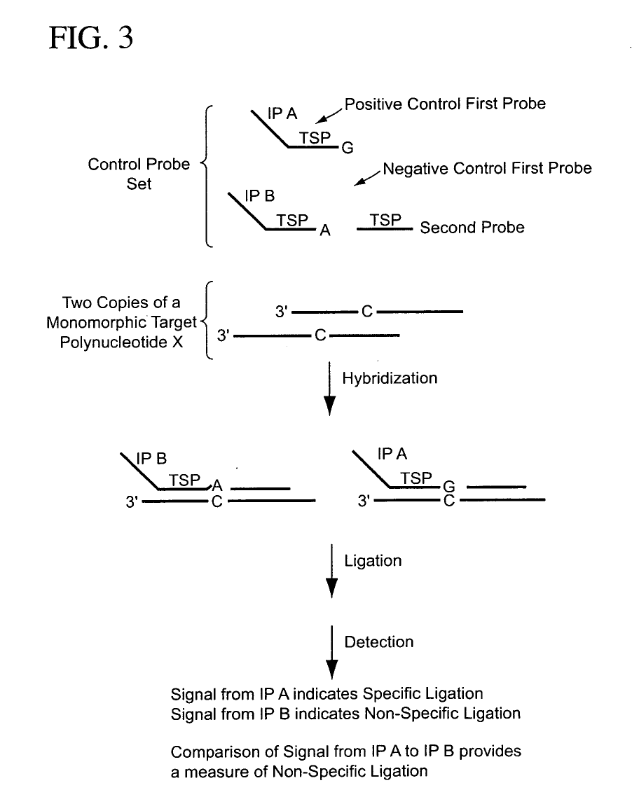

Some embodiments of the present teachings pertain to methods of detecting specific ligation and non-specific ligation in a ligation assay (see for FIG. 34). As depicted in FIG. 3, a positive control first probe one and a negative control first probe one can each comprise target specific portions that can hybridize to a monomorphic target polynucleotide sequence X. The target specific portion of the negative control first probe one can further comprise a discriminating region (here, an A), that does not hybridize with the corresponding nucleotide of the monomorphic target polynucleotide, and the target specific portion of the positive control first probe one can further comprise a discriminating region (here, a G) that does hybridize with the corresponding nucleotide of the monomorphic target polynucleotide. The positive control first probe and the negative control first probe can further comprise different identifying portions (here, IP A for the positive control first probe one, and IP B for the negative control first probe one). The positive control first probe one and negative control first probe one, and a second probe, can hybridize adjacently on a region of the monomorphic target polynucleotide sequence. Following hybridization of the positive control first probe one and the second probe to the monomorphic target polynucleotide sequence, ligation can occur, resulting in a specific ligation product comprising the positive control first probe one and the second probe. Following hybridization of the negative control first probe one and the second probe to the monomorphic target polynucleotide sequence, non-specific ligation can occur, resulting in a non-specific ligation product comprising the negative control first probe one and the second probe. Detection of the ligation products comprising the IP A of the positive control first probe one can result in the production of a distinct signal indicating the occurrence of specific ligation. Detection of the ligation products comprising the IP B of the negative control fist probe can result in the production of a distinct signal indicating the occurrence of non-specific ligation. By comparing the signal from the positive control first probe one product to the signal from the negative control first probe one product, the experimentalist can acquire an indication of the degree of specificity within the ligation reaction.

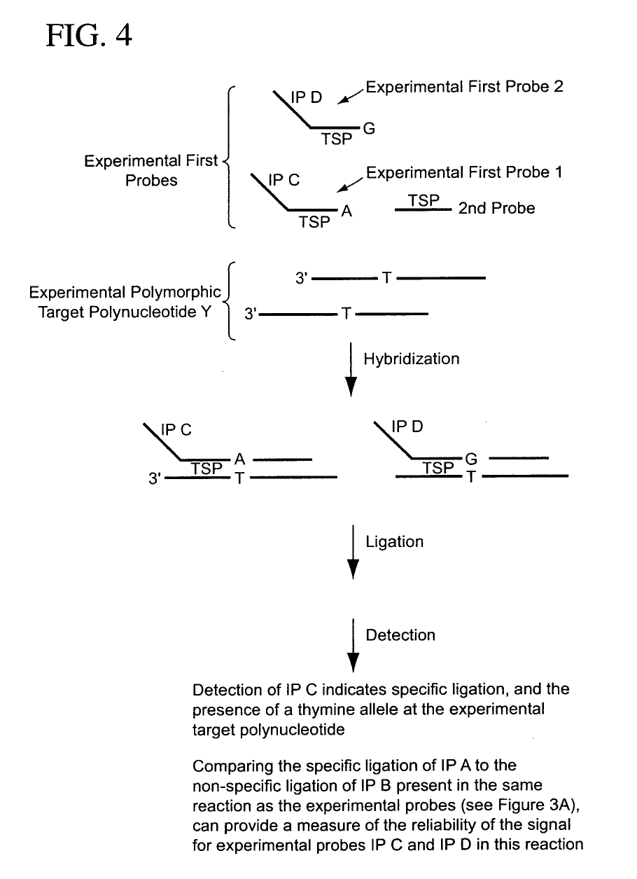

As depicted in FIG. 4, the control probe reactions querying monomorphic target polynucleotide sequences (for example polynucleotide X in FIG. 3) can occur in the same reaction as experimental probe reactions querying polymorphic target polynucleotide sequences (for example Y in FIG. 4). An experimental first probe one and an experimental first probe two can each comprise target specific portions that can hybridize to a polymorphic morphic target polynucleotide sequence. The target specific portion of the experimental first probe one can further comprise a discriminating region that can hybridize with the corresponding nucleotide of the polymorphic target polynucleotide (here, an A), and the target specific portion of the experimental first probe two can further comprise a discriminating region that does not hybridize with the corresponding nucleotide of the polymorphic target polynucleotide (here, a G). The experimental first probe one and the experimental first probe two can further comprise different identifying portions (here, IP C for experimental first probe one, and IP D for the experimental first probe two). The experimental first probe one and experimental first probe two, can hybridize adjacently the experimental second probe on the polymorphic target polynucleotide sequence. Following hybridization of the experimental first probe one and the second probe to the monomorphic target polynucleotide sequence, ligation can occur, resulting in a specific ligation product comprising the positive control first probe one and the second probe. Following hybridization of the experimental first probe two and the second probe to the polymorphic target polynucleotide sequence, non-specific ligation can occur, resulting in a non-specific ligation product comprising the experimental first probe two and the second probe. Detection of the ligation products comprising the IP C of the experimental first probe one can result in the production of a distinct signal indicating the occurrence of specific ligation. Detection of the ligation products comprising the IP D of the experimental fist probe two can result in the production of a distinct signal indicating the occurrence of non-specific ligation. By comparing the signal from the experimental first probe one product to the signal from the experimental first probe two product, the experimentalist can acquire an indication of the degree of specificity in the ligation reaction. Further, by comparing the difference between the positive control probe product and the negative control probe product, to the difference between the experimental probe one product and the experimental probe two product, the experimentalist can acquire an indication of the likelihood that signal originating from experimental probe two indicates a non-specific ligation product rather than a specific ligation product, thereby providing a way of measuring the confidence in obtaining an accurate assessment of the identity of the polymorphic target polynucleotide sequence.

In some embodiments, the polymorphic target polynucleotide sequence further comprises different single nucleotide polymorphism (SNP) variants of a particular genomic locus (for example, a gene). The target specific portion of each experimental first probe can comprise a discriminating region that is complementary to a particular allelic variant. Following hybridization and ligation, detection and quantification of the identifying portion of the ligation product or the ligation product surrogate comprising the experimental probes can result in the identification of the SNP. Further, detection and quantification of the identifying portion of the ligation product or ligation product surrogate comprising the control probes can result in a determination of the extent of specific and non-specific ligation, thereby providing a way of measuring the confidence in obtaining an accurate assessment of the identity of the polymorphic target polynucleotide sequence.

Some embodiments comprise a plurality of experimental probe sets in a ligation assay querying a plurality of polymorphic target polynucleotide sequences occurring in the same reaction as at least one control probe set querying at least one monomorphic target polynucleotide sequence. In some embodiments, a plurality of experimental probe in a ligation assay query a plurality of SNP loci, wherein each SNP locus can comprise polymorphic allelic variants comprising single nucleotide polymorphisms. In some embodiments, between 1 and 10 polymorphic target polynucleotide sequences are queried. In some embodiments, between 1 and 50 polymorphic target polynucleotide sequences are queried. In some embodiments, between 1 and 100 polymorphic target polynucleotide sequences are queried. In some embodiments, between 1 and 200 polymorphic target polynucleotide sequences are queried. In some embodiments, greater than 200 polymorphic target polynucleotide sequences are queried.

In some embodiments, between 1 and 10 polymorphic target polynucleotide sequences are queried in a reaction with at least one control probe set. In some embodiments, between 1 and 50 polymorphic target polynucleotide sequences are queried in a reaction with one control probe set. In some embodiments, between 1 and 100 polymorphic target polynucleotide sequences are queried in a reaction with one control probe set. In some embodiments, between 1 and 200 polymorphic target polynucleotide sequences are queried in a reaction with one control probe set. In some embodiments, greater than 200 polymorphic target polynucleotide sequences are queried in a reaction with one control probe set.

In some embodiments, between 1 and 10 polymorphic target polynucleotide sequences are queried in a reaction with two control probe sets. In some embodiments, between 1 and 50 polymorphic target polynucleotide sequences are queried in a reaction with two control probe sets. In some embodiments, between 1 and 100 polymorphic target polynucleotide sequences are queried in a reaction with two control probe sets. In some embodiments, between 1 and 200 polymorphic target polynucleotide sequences are queried in a reaction with two control probe sets. In some embodiments, greater than 200 polymorphic target polynucleotide sequences are queried in a reaction with at two control probe sets.

In some embodiments, between 1 and 10 polymorphic target polynucleotide sequences are queried in a reaction with at least three control probe sets. In some embodiments, between 1 and 50 polymorphic target polynucleotide sequences are queried in a reaction with at least three control probe sets. In some embodiments, between 1 and 100 polymorphic target polynucleotide sequences are queried in a reaction with at least three control probe sets. In some embodiments, between 1 and 200 polymorphic target polynucleotide sequences are queried in a reaction with at least three control probe sets. In some embodiments, greater than 200 polymorphic target polynucleotide sequences are queried in a reaction with at least three control probe sets.

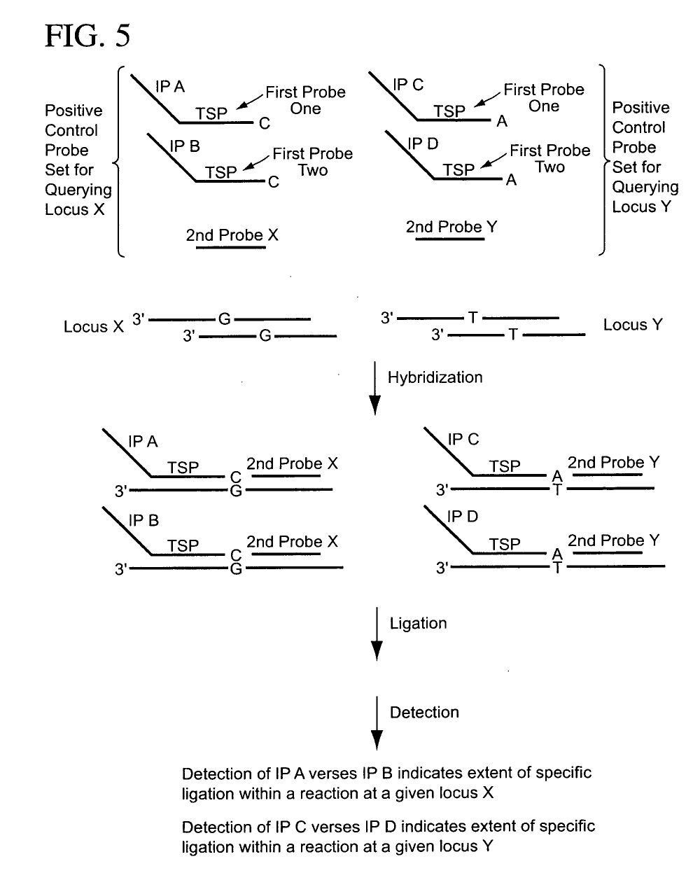

In some embodiments of the present teachings, at least two monomorphic target polynucleotides are queried in a first reaction with at least two positive control probe sets (as depicted in FIG. 5). Comparing the products resulting from a reaction comprising a first positive control probe set querying locus X and a second positive control probe set querying locus Y can provide a measure of the extent to which specific ligation varies for different monomorphic target polynucleotide sequences within a given reaction. Comparing the products resulting from a first reaction comprising a first positive control probe set querying locus X and a second positive control probe set querying locus Y in a first reaction, to the products resulting from a second reaction comprising a first positive control probe set querying locus X and a second positive control probe set querying locus Y can provide a measure of the extent to which specific ligation varies for the same monomorphic target polynucleotide sequences between different reactions.

FIG. 5 depicts a reaction comprising a positive control probe set for querying a locus X, and a positive control probe set for querying a locus Y. The positive control probe set for querying locus X comprises a positive control first probe one and a positive control first probe two, each comprising identical target specific portions that can hybridize to a monomorphic target polynucleotide sequence (locus X). The positive control first probe one comprises an identifying portion A (IP A) that differs from the identifying portion for positive control first probe two (here, IP B), however both positive control first probe one and positive control first probe two of the positive control probe set querying locus X comprise the same 3′ discriminating region (here a C). The positive control probe set for querying locus Y comprises a positive control first probe one and a positive control first probe two, each comprising identical target specific portions that can hybridize to a monomorphic target polynucleotide sequence (locus Y). The positive control first probe one comprises an identifying portion C (IP C) that differs from the identifying portion for positive control first probe two (here, IP D), however both positive control first probe one and positive control first probe two of the positive control probe set querying locus Y comprise the same 3′ discriminating region (here an A).

The positive control first probes and the second probes can hybridize to their corresponding monomorphic target polynucleotide sequence wherein the discriminating region hybridizes to the corresponding nucleotide on the monomorphic target polynucleotide sequence, thereby allowing the positive control first probes to hybridize to their corresponding monomorphic target polynucleotide. Following hybridization of the positive control first probes and the positive control second probes to the monomorphic target polynucleotide sequence, a ligation agent can be provided, thereby allowing ligation to occur, resulting in specific ligation products from the positive control probe set querying locus X comprising the first positive control probe one and the second probe, and the first positive control probe two and the second probe, as well as specific ligation products from the positive control probe set querying locus Y comprising the first positive control probe one and the second probe, and the first positive control probe two and the second probe. Detection of IP A and IP B in the ligation products can result in the production of two distinct signals from a monomorphic target polynucleotide, and a measure of specific ligation. Detection of IP C and IP D in the ligation products can result in the production of two distinct signals from a monomorphic target polynucleotide, and a measure of specific ligation. Comparison of the signal produced from IP A to IP B can provide a measure of specific ligation at a target polynucleotide (here locus X) within a reaction. Comparison of the signal produced from IP A and IP B to the signal produced from IP C and IP D can provide a measure of specific ligation at different target polynucleotides (here locus X and locus Y) within a reaction.

In some embodiments, a parallel reaction comprising the same positive control set querying locus X and the positive control set querying locus Y, and the same monomorphic target polynucleotides (locus X and locus Y), can provide a measure of specific ligation at a given locus or loci across reactions.

In some embodiments, between 1 and 10 monomorphic target polynucleotide sequences are queried in a reaction comprising between 1 and 10 positive control probe sets. In some embodiments, between 10 and 50 monomorphic target polynucleotide sequences are queried in a reaction comprising between 10 and 50 positive control probe sets. In some embodiments, between 50 and 100 monomorphic target polynucleotide sequences are queried in a reaction comprising between 50 and 100 positive control probe sets. In some embodiments, 48 monomorphic target polynucleotide sequences are queried in a reaction comprising 48 positive control sets. In some embodiments, 96 monomorphic target polynucleotide sequences are queried in a reaction comprising 96 positive control probe sets. In some embodiments, 192 monomorphic polynucleotide sequences are queried in a reaction comprising 192 positive control probe sets. In some embodiments, greater than 192 monomorphic target polynucleotide sequences are queried in a reaction comprising greater than 192 positive control sets. It will be appreciated that any and all of these reaction scenarios, as well as others, can be performed with parallel reactions concurrently. In some embodiments, the parallel reactions can comprise the same positive control probe sets and target polynucleotide sequences. In some embodiments, the parallel reactions can comprise different positive control probe sets and different target polynucleotide sequences. In some embodiments, the parallel reactions can comprise negative control probe sets (see infra) querying the same target polynucleotides as the positive control probe sets. In some embodiments, the parallel reactions can comprise negative control probe sets (see infra) querying different target polynucleotides as the positive control probes sets.

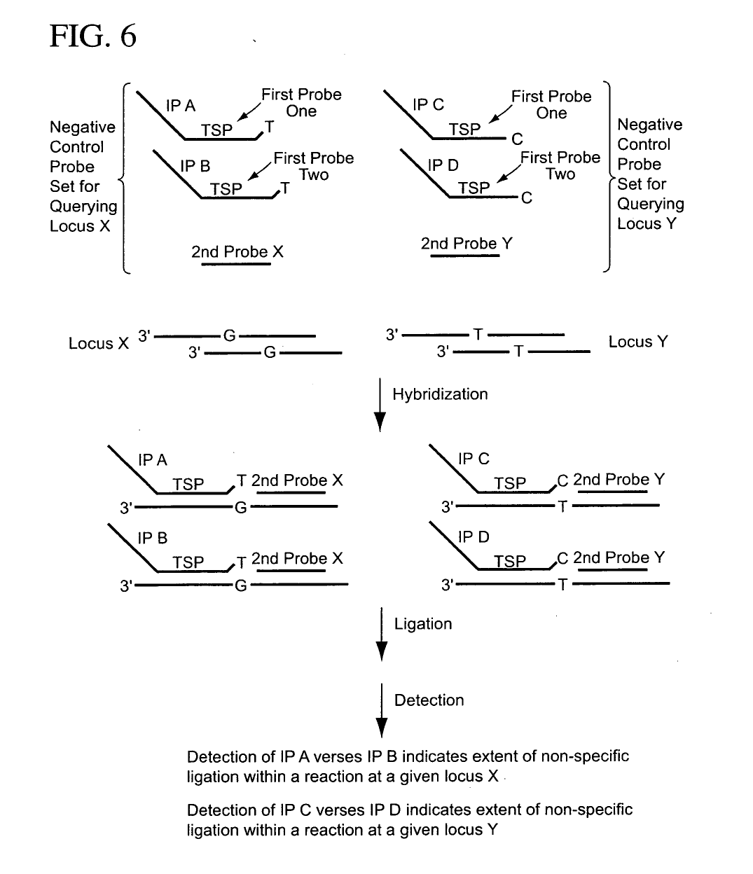

In some embodiments of the present teachings, at least two monomorphic target polynucleotides are queried in a reaction with at least two negative control probe sets (as depicted in FIG. 6). Comparing the products resulting from a reaction comprising a first negative control probe set querying locus X and a second negative control probe set querying locus Y can provide a measure of the extent to which non-specific ligation varies for different monomorphic target polynucleotide sequences within a given reaction. Comparing the products resulting from a first reaction comprising a first negative control probe set querying locus X and a second negative control probe set querying locus Y in a first reaction, to the products resulting from a second reaction comprising a first negative control probe set querying locus X and a second negative control probe set querying locus Y can provide a measure of the extent to which non-specific ligation varies for the same monomorphic target polynucleotide sequences between different reactions.

FIG. 6 depicts a reaction comprising a negative control probe set for querying a locus X, and a negative control probe set for querying a locus Y. The negative control probe set for querying locus X comprises a negative control first probe one and a negative control first probe two, each comprising identical target specific portions that can hybridize to a monomorphic target polynucleotide sequence (locus X), as well as identical discriminating regions (here, a T) of the target specific portion that are not complementary to the corresponding nucleotide (here, a G) on the target monomorphic polynucleotide (locus X). The negative control first probe one comprises an identifying portion A (here, IP A) that differs from the identifying portion for negative control first probe two (here, IP B). The negative control probe set for querying locus Y comprises a negative control first probe one and a negative control first probe two, each comprising identical target specific portions that can hybridize to a monomorphic target polynucleotide sequence (locus Y), as well as identical discriminating regions (here, a C) of the target specific portion that are not complementary to the corresponding nucleotide (here, a T) on the target monomorphic polynucleotide (locus Y). The negative control first probe one comprises an identifying portion C (here, IP C) that differs from the identifying portion for negative control first probe two (here, IP D).

The negative control first probes and the second probes can hybridize to their corresponding monomorphic target polynucleotide sequence wherein the discriminating region is not complementary to the corresponding nucleotide on the monomorphic target polynucleotide sequence, thereby preventing the negative control first probes from completely hybridizing to their corresponding monomorphic target polynucleotide. Following hybridization of the negative control first probes and the negative control second probes to the monomorphic target polynucleotide sequence, a ligation agent can be provided, thereby allowing non-ligation to occur, resulting in non-specific ligation products from the negative control probe set querying locus X comprising the first negative control probe one and the second probe, and the first negative control probe two and the second probe, as well as non-specific ligation products from the negative control probe set querying locus Y comprising the first negative control probe one and the second probe, and the first negative control probe two and the second probe. Detection of IP A and IP B in the ligation products can result in the production of two distinct signals from a monomorphic target polynucleotide, and a measure of non-specific ligation. Detection of IP C and IP D in the ligation products can result in the production of two distinct signals from a monomorphic target polynucleotide, and a measure of non-specific ligation. Comparison of the signal produced from IP A to IP B can provide a measure of non-specific ligation at a target polynucleotide (here locus X) within a reaction. Comparison of the signal produced from IP A and IP B to the signal produced from IP C and IP D can provide a measure of non-specific ligation at different target polynucleotides (here locus X and locus Y) within a reaction.

In some embodiments, a parallel reaction comprising the same negative control set querying locus X and the negative control set querying locus Y, and the same monomorphic target polynucleotides (locus X and locus Y), can provide a measure of specific ligation at a given locus or loci across reactions.

In some embodiments, between 1 and 10 monomorphic target polynucleotide sequences are queried in a reaction comprising between 1 and 10 negative control probe sets. In some embodiments, between 10 and 50 monomorphic target polynucleotide sequences are queried in a reaction comprising between 10 and 50 negative control probe sets. In some embodiments, between 50 and 100 monomorphic target polynucleotide sequences are queried in a reaction comprising between 50 and 100 negative control probe sets. In some embodiments, 48 monomorphic target polynucleotide sequences are queried in a reaction comprising 48 negative control sets. In some embodiments, 96 monomorphic target polynucleotide sequences are queried in a reaction comprising 96 negative control probe sets. In some embodiments, 192 monomorphic polynucleotide sequences are queried in a reaction comprising 192 negative control probe sets. In some embodiments, greater than 192 monomorphic target polynucleotide sequences are queried in a reaction comprising greater than 192 negative control sets. It will be appreciated that any and all of these reaction scenarios, as well as others, can be performed with parallel reactions concurrently. In some embodiments, the parallel reactions can comprise the same negative control probe sets and target polynucleotide sequences. In some embodiments, the parallel reactions can comprise different negative control probe sets and different target polynucleotide sequences. In some embodiments, the parallel reactions can comprise positive control probe sets (see supra) querying the same target polynucleotides as the negative control probe sets. In some embodiments, the parallel reactions can comprise positive control probe sets (see supra) querying different target polynucleotides as the negative control probes sets.

In some embodiments of the present teachings, a plurality of monomorphic target polynucleotide sequences are queried in parallel reactions, wherein a plurality of monomorphic target polynucleotide sequences are queried in a first reaction with a plurality of positive control probe sets, and wherein a plurality of monomorphic target polynucleotide sequences are queried in a second reaction with a plurality of negative control probe sets. In some embodiments, a comparison of the extent of non-specific ligation in the reaction comprising negative control probe sets to the extent of specific ligation in the reaction comprising positive control probes provides a measure of the extent non-specific ligation is occurring across different reactions.

In some embodiments, measures of non-specific ligation acquired with negative control probes can be compared to parallel reactions comprising polymorphic target polymorphic polynucleotides that are queried by experimental probes, and thereby provide an assessment of the likelihood of specific and non-specific ligation in the parallel reaction comprising experimental probes. In some embodiments, measures of non-specific ligation acquired with negative control probes can be compared to other non-parallel reactions comprising polymorphic target polymorphic polynucleotides that are queried by experimental probes, and thereby provide an assessment of the likelihood of specific and non-specific ligation in the non-parallel reaction comprising experimental probes.

In some embodiments, the monomorphic target polynucleotide sequences in a first reaction comprising positive control probe sets are the same as the monomorphic target polynucleotide sequences queried in a parallel second reaction comprising negative control probe sets. In some embodiments, the monomorphic target polynucleotide sequences queried in a first reaction comprising positive control probe sets are different from the monomorphic target polynucleotide sequences queried in a second reaction comprising negative control probe sets. In some embodiments, some of the monomorphic target polynucleotide sequences queried in a first reaction comprising positive control probe sets are the same as some of the monomorphic target polynucleotide sequences queried in a second reaction comprising negative control probe sets, whereas some of the monomorphic target polynucleotide sequences queried in the first reaction comprising positive control probe sets are different from some of the monomorphic target polynucleotide sequences queried in the second reaction comprising negative control probe sets.

In some embodiments, the monomorphic target polynucleotide sequences in a first reaction comprising positive control probe sets are the same as the monomorphic target polynucleotide sequences queried in a second reaction comprising negative control probe sets, and further the identifying portions of the positive control probe sets of the first reaction are the same as the identifying portions of the negative control probe sets of the second reaction. In some embodiments, the monomorphic target polynucleotide sequences in a first reaction comprising positive control probe sets are the same as the monomorphic target polynucleotide sequences queried in a second reaction comprising negative control probes, and further the identifying portions of the positive control probe sets of the first reaction are different from the identifying portions of the negative control probe sets of the second reaction. In some embodiments, the monomorphic target polynucleotide sequences in a first reaction comprising positive control probe sets are the same as the monomorphic target polynucleotide sequences queried in a second reaction comprising negative control probe sets, and further some but not all of the identifying portions of the positive control probe sets of the first reaction are different from the identifying portions of the negative control probe sets of the second reaction.

It will thus be appreciated that various permutations of monomorphic target polynucleotide sequences between reactions comprising positive control probe sets and reactions comprising negative control probe sets, and the extent to which the identifying portions of the positive control probes and negative control probes are all the same, all different, or the same and different, can vary and nonetheless remain within the scope of the present teachings.

It will also be appreciated that various permutations of monomorphic target polynucleotide sequences between reactions comprising positive control probe sets and reactions comprising negative control probe sets, and the extent to which the target monomorphic sequences are all the same, all different, or the same and different, can vary and nonetheless remain within the scope of the present teachings.

It will be appreciated that various permutations of monomorphic target polynucleotide sequences between reactions comprising positive control probe sets and reactions comprising negative control probe sets, and the extent to which the identifying portions of the positive control probes and negative control probes are all the same, all different, or the same and different, can vary and nonetheless remain within the scope of the present teachings, and the extent to which the target monomorphic polynucleotide sequences of the positive control reactions and the negative control reactions are all the same, all different, or the same and different can vary, as well as combinations thereof between identifying portions and target monomorphic polynucleotides, and nonetheless remain with the scope of the present teachings.

In some embodiments of the present teachings, the ligation reaction can be preceeded by a whole genome amplification reaction.

In some embodiments of the present teachings, the experimental first probes and/or control first probes and/or experimental second probes and/or control second probes can comprise looped linker compositions, and/or non-looped linker compositions, as described for example in U.S. Provisional Application 60/517,470. In some embodiments of the present teachings, mobility probes are hybridized to the identifier portion (or identifier portion complements) of the ligation products or ligation product surrogates, and the identity of the target determined from the eluted mobility probe in a mobility-dependent analysis technique as taught for example in P.C.T. Application U.S. 200337227.

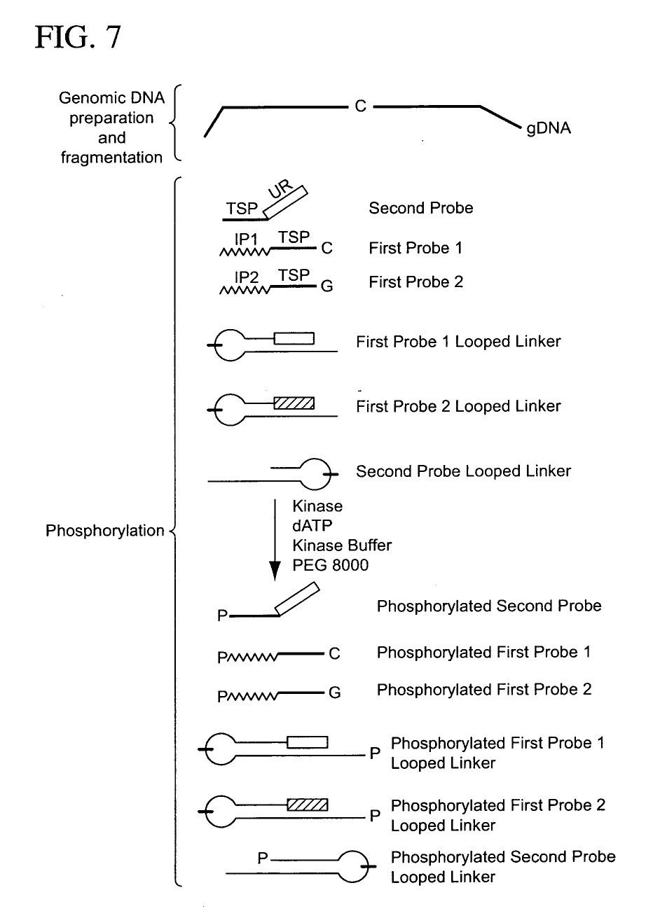

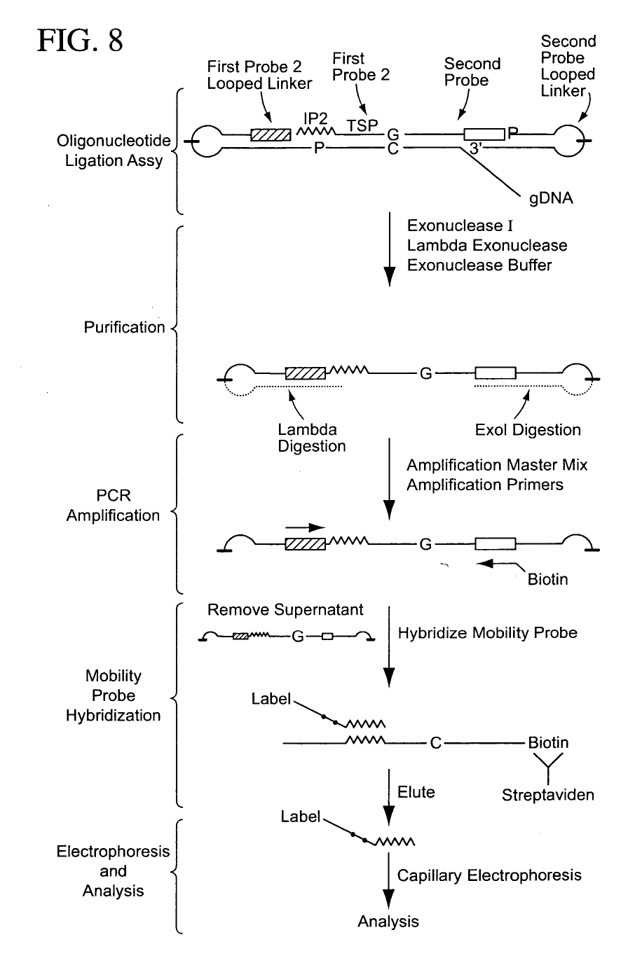

In some embodiments comprising looped linkers, a variety of probes can first be phosphorylated (see illustration of various species in FIG. 7). The phosphorylated probes can then be employed in a ligation reaction according to some embodiments of the present teachings as depicted schematically in FIG. 8. For example, a second probe looped linker can be considered downstream (located 3′) to a second probe. The second probe looped linker comprises a 3′ single stranded PCR universal reverse priming portion, an internal blocking moiety (shown in FIGS. 7 and 8 as a horizontal line through the middle of the loop), and a 5′ double stranded PCR universal reverse priming portion. The single stranded portion of a second probe looped linker can anneal with a universal reverse priming portion of the second probe, thereby allowing ligation of the universal reverse priming region of the looped linker to the universal reverse priming portion of the second probe. Further, a first probe looped linker can be considered upstream (located 5′) to a first probe. The first probe looped linker further comprises a 3′ double stranded PCR universal forward priming portion, an internal blocking moiety, and a 5′ single stranded partial identifying portion 2. The first probe two looped linker can anneal with the identifying portion 2 of the first probe 2, thereby allowing ligation of the universal forward priming portion of the first probe looped linker to the target identifying portion of the first probe.

In some embodiments comprising looped linkers (see for illustration FIG. 8, left side) the looped linker of the first probe can further comprise an internally located blocking moiety, which can impart varying degrees of resistance to nuclease digestion, depending on whether, and what kind of, ligation product it is incorporated into. For example, first probe looped linkers that are incorporated into concatameric ligation products can be sensitive to 5′-acting nuclease digestion proceeding from their 5′ ends to the blocking moiety, thereby allowing for the generation of a single stranded area on which a PCR primer can eventually hybridize. Moreover, first probe looped linkers that are not incorporated into concatameric ligation products are sensitive to both 5′phosphate-acting nuclease digestion proceeding from their 5′ phosphate ends to the blocking moiety, as well as sensitive to 3′-acting nuclease digestion proceeding from their free 3′ ends. Further, first probe looped linkers that are ligated to ASO's, but that are not incorporated into a complete ligation product are also sensitive to both 3′-acting degradation via the first probe, as well as directly via 5′phosphate-acting nucleases.

In some embodiments comprising looped linkers (see for illustration FIG. 8, right side), the second probe looped linker can comprise an internally located blocking moiety, which can impart varying degrees of resistance to nuclease digestion depending on whether, and what kind of, ligation product it is incorporated into. For example, second probe looped linkers that are incorporated into ligation products can be sensitive to 3′-acting nuclease digestion proceeding from their 3′ ends to the blocking moiety, thereby allowing for the generation of a single stranded area on which a PCR primer can eventually hybridize. Moreover, second probe looped linkers that are not incorporated into concatameric ligation products are sensitive to both 5′ phosphate-acting nuclease digestion proceeding from their 5′ phospate ends to the blocking moiety, as well as sensitive to 3′-acting nuclease digestion proceeding from their free 3′ ends to the blocking moiety. Further, second probe looped linkers that are ligated to second probes, but that are not incorporated into a full ligation product are also sensitive to 3′-acting nucleases directly, as well 5′-acting nucleases via degradation through the second probe. Removal of incorporated reaction components can facilitate downstream reactions, such as PCR.

In some embodiments comprising looped linkers, exemplary blocking moieties comprise C3, C9, C12, and C18, available commercially from Glen Research, tetra methoxy uracil, as well as moieties described for example in U.S. Pat. No. 5,514,543, and Woo et al., U.S. patent application Ser. No. 09/836,704. Exemplary nucleases comprise exonuclease 1 and lambda exonuclease, which act on the 3′ and 5′ phosphate ends, respectfully, of single stranded oligonucleotides. Other enzymes as appropriate for practicing the present teachings are further contemplated, and are commercially available from such sources as New England Biolabs, Roche, and Stratagene.

In some embodiments of the present teachings, a second ligation reaction can introduce the mobility probe as taught for example in U.S. Provisional Application 60/477,614.

In some embodiments of the present teachings, the ligation reaction can be performed concurrently with, for example, a decontamination reaction and/or a phosphorylation reaction. In some embodiments of the present teachings, the ligation reaction can be performed concurrently with, for example, a first decontamination reaction and/or a phosphorylation reaction, followed by the ligation, wherein the ligase is a heat-activate-able ligase. For further illustrative teachings of such approaches, see for example U.S. Provisional Application 60/584,682 Methods, Reaction Mixtures, and Kits for Ligating Polynucleotides to Andersen et al., and co-filed non-provisional application claiming priority thereto.

In some embodiments of the present teachings, the control probes and/or experimental probes further comprise a primer portion, and the ligation reaction is followed by an amplification reaction. In some embodiments, the amplification reaction is a PCR. In some embodiments, the primer portions in the control first probes and/or experimental first probes can comprise a forward universal primer portion. In some embodiments, the primer portions in the control second probes and/or experimental second probes can comprise a reverse universal primer portion, such that a single set of universal primers can amplify all the ligation products resulting from the ligation products of the experimental first probes to the experimental second probes as well as ligation products of the control first probes to the control second probes. In some embodiments, the primer portions in the control probes and/or experimental probes can comprise a plurality of universal primer portion sequences, such that a single battery of universal primers can amplify all the ligation products resulting from the ligation product of the experimental probe sets as well as the ligation products of the control probe sets. In some embodiments, the control probe sets of the present teachings can be employed in the context of various ligation-mediated encoding and decoding strategies for detecting target polynucleotides employing batteries of universal address primer sets, as discussed for example in U.S. Non-Provisional Patent Applications 10/090,830 to Chen et al., and 11/090,468 to Lao et al.,

In some embodiments of the present teachings, control and experimental ligation products are amplified by a PCR, the resulting amplicons hybridized with mobility probes, and the identity of the target polynucleotide determined therefrom based on the identity of the identifying portion. In some embodiments of the present teachings, at least one monomorphic target polynucleotide sequence is queried in a reaction comprising control probes along with a plurality of polymorphic target polynucleotide sequences and their corresponding experimental probes. In some embodiments, the ligation products resulting from the monomorphic target polynucleotide sequence and the ligation products resulting from the polymorphic target polynucleotide sequence are hybridized with mobility probes complementary to the identifying portions (or identifying portion complements) introduced by the probes in the ligation reaction. In some embodiments, some of the mobility probes included in the hybridization reaction do not hybridize with identifying portions found in any of the ligation products and/or ligation product surrogates. Including such mobility probes that do not correspond to identifying portions included in the probes of the ligation reaction can provide, for example, ease of procedural steps in a highly multiplexed assay.

In some embodiments, universal bases can be incorporated into the target specific portion of first probes and/or second probes, for example when undesirable polymorphisms are present in the putative monomorphic target polynucleotide sequence. Such universal bases can, for example, affect the ability of the first probes and/or the second probes, to hybridize to the putative monomorphic target polynucleotide sequence to achieve the desired hybridization in accordance with some embodiments of the present teachings. In some embodiments, universal bases can be incorporated into the target specific portion of experimental probes that query a polymorphic target polynucleotide sequence, thereby providing the ability of the experimental probes to hybridize to the polymorphic target polynucleotide sequence to achieve the desired hybridization in accordance with some embodiments of the present teachings. For example, universal nucleobases can be incorporated into probes to account for polymorphisms close to the polymorphism of interest, thereby allowing a probe to query the polymorphism of interest without the complication of the nearby polymorphism, since the probe's universal base can hybridize to the nearby polymorphism in a manner independent of its identity.

In some embodiments of multiplexed ligation reactions, the present teachings can be practiced with a plurality of primers corresponding to the identifying portion of the ligation probes. For example, in a SNP detection context, the ligation probe one and ligation probe corresponding to the two allelic variants of a SNP locus can vary. The second probe can comprise a universal primer portion. After the ligation reaction, a PCR can be performed wherein the primers in the PCR comprise a forward PCR corresponding to the identifying portion of ligation probe one, a forward PCR primer corresponding to the identifying portion of ligation probe two, and a universal reverse primer corresponding to the universal primer portion of the second probe. Such approaches can serve to reduce the length of the first ligation probes.

In some embodiments of multiplexed ligation reactions, the present teachings can be practiced with a plurality of primers corresponding to the partial identifying portion of the ligation probes. For example, in a SNP detection context, the ligation probe one and ligation probe corresponding to the two allelic variants of a SNP locus can have partial identifying portions that vary. The second probe can comprise a partial universal primer portion. After the ligation reaction, a PCR can be performed wherein the primers in the PCR comprise a forward PCR corresponding to the partial identifying portion of ligation probe one as well as additional identifying portion sequence for probe one, a forward PCR primer corresponding to the partial identifying portion of ligation probe two as well as additional identifying portion sequence for probe two, and a universal reverse primer corresponding to the partial universal primer portion of the second probe as well as additional identifying portion sequence for probe two. As a result, the PCR results in the full identifying portion being present in the products. Detection with, for example, mobility probes comprising the full identifying portions can then be performed.

In some embodiments, the primer sequences corresponding to the identifying portions (or partial identifying portions) can additionally comprise the same 5′ universal primer sequence. Such an approach can reduce the concern that in highly multiplexed amplification reactions primer-dimers will form in excess due to the large number of different identifying portion-based primers. The universal primer sequence can be designed to have a higher Tm than the identifying portion primer sequence. With both universal primers and identifying portion primers in the amplification reaction, an initial few cycles (2-4, for example) can be performed at a lower assay temperature at which the identifying portion primer will anneal to and extend the reverse strand of the ligation product. Thereafter, the assay temperature can be increased to push the reaction towards the use of the universal primer.

Kits

Kits for assessing ligation of at least one target polynucleotide are also provided in the present teachings. In some embodiments, kits serve to expedite the performance of the disclosed methods by assembling two or more components required for carrying out the methods. In some embodiments, kits generally contain components in pre-measured unit amounts to minimize the need for measurements by end-users. In some embodiments, kits preferably include instructions for performing one or more of the disclosed methods. In some embodiments, the kit components are optimized to operate in conjunction with one another.

In some embodiments, a positive control ligation kit can comprise a positive control probe set, an experimental probe set, a ligation agent, a target polynucleotide, and combinations thereof. In some embodiments, a negative control ligation kit can comprise a negative control probe set, an experimental probe set, a ligation agent, a target polynucleotide, and combinations thereof. In some embodiments, a control ligation kit can comprise a negative control probe set, a positive control probe set, an experimental probe set, a ligation agent, a target polynucleotide, and combinations thereof. In some embodiments, an experimental ligation kit can comprise an experimental probe set, a ligation agent, a target polynucleotide, and combinations thereof. In some embodiments, an amplification kit comprises a primer, an affinity moiety primer, a polymerase, and nucleotides. In some embodiments, a purification kit comprises a nuclease, a glycosylase, and combinations thereof. In some embodiments, a PCR purification kit comprises an affinity-moiety binder and a solid support. In some embodiments, a phosphorylation kit comprises a kinase. In some embodiments, a mobility probe kit comprises a mobility probe.

In some embodiments, kits can comprise none, some, or all of a positive control ligation kit, a negative control ligation kit, a control ligation kit, an experimental ligation kit, an amplification kit, a purification kit, a PCR purification kit, a phosphorylation kit, a mobility probe kit, and combinations thereof.

In some embodiments, kits are disclosed that comprise at least one means for ligating, at least one means for amplifying, at least one means for removing unincorporated and/or unwanted reaction components, at least one means for detecting, and combinations thereof. In some embodiments, kit configurations in accordance with the present teachings can be found for example in the Applied Biosystems SNPlex™ Genotyping System Chemistry Guide.

While the present teachings have been described in terms of these examples and exemplary embodiments, the skilled artisan will readily understand that numerous variations and modifications of these exemplary embodiments are possible without undue experimentation. All such variations and modifications are within the scope of the current teachings.

We claim:

1. A method for determining ligation in a ligation assay comprising;
providing a monomorphic target polynucleotide sequence and a control probe set, wherein the control probe set comprises a positive control first probe and a negative control first probe, and a second probe,
wherein the positive control first probe comprises a target specific portion, wherein the target specific portion comprises a discriminating region, and an identifying portion,
wherein the negative control first probe comprises a target specific portion,
wherein the target specific portion comprises a discriminating region, and an identifying portion,
wherein the identifying portion of the positive control first probe and the negative control first probe are different,
wherein the second probe of the control probe set comprises a target specific portion;
hybridizing the first positive control probe, the first negative control probe, and the second probe to the monomorphic target polynucleotide sequence, wherein the positive control probe and the negative control probes can hybridize adjacently to the second probe on the monomorphic target polynucleotide sequence, wherein the discriminating region of the positive control probe allows complete complementarity with the monomorphic target polynucleotide, wherein the discriminating region of the negative control probes prevents complete complementarity with the monomorphic target polynucleotide;
ligating the positive control first probe to the second probe, ligating the negative control first probe to the second probe, thereby generating a specific ligation product and a non-specific ligation product comprising different identifying portions;
separating the non-specific ligation products and the specific ligation products from the unhybridized control probes and unligated control probes;
detecting the specific and non-specific ligation products based on their distinct identifying portions;
comparing the amount of specific ligation products to non-specific ligation products;
thereby assessing ligation in a ligation assay.

2. The method according to claim 1 further comprising a polymorphic polynucleotide target sequence and an experimental probe set, wherein the experimental probe set comprises an experimental first probe one, an experimental first probe two, and an experimental second probe,
wherein the experimental first probe one comprises an identifying portion and a target specific portion complementary to the polymorphic polynucleotide target sequence, wherein the target specific portion comprises a discriminating region,
wherein the experimental first probe two comprises an identifying portion and a target specific portion complementary to the polymorphic polynucleotide target sequence,
wherein the target specific portion comprises a discriminating region,
wherein the identifying portion of the experimental first probe one differs from the identifying portion of the experimental first probe two,
wherein the discriminating region of the experimental first probe one differs from the discriminating region of the experimental first probe two,
wherein the second experimental probe comprises a target specific portion complementary to the polymorphic polynucleotide target sequence,
wherein the discriminating region of the experimental first probes can hybridize with different nucleotides corresponding to different alleles of a single nucleotide polymorphism,
wherein the experimental first probes are hybridized adjacent to the experimental second probe on the polymorphic target polynucleotide sequence;
ligating the experimental first probe one to the contiguously hybridized experimental second probe, ligating the experimental first probe two to the contiguously hybridized second probe, thereby generating specific ligation products comprising different identifying portions corresponding to two alleles of a single nucleotide polymorphism;
separating the specific ligation products and the non-specific ligation products from the unhybridized experimental first probes and experimental second probes, and unligated experimental first probes and experimental second probes;
detecting the specific and non-specific ligation products based on their distinct identifying portions;
comparing the amount of specific ligation products to non-specific ligation products resulting from the control probe set;
comparing the ligation products resulting from the control probe set to the ligation products resulting from the experimental probe set.

3. The method according to claim 2 wherein the ligation products are amplified by a PCR.

4. The method according to claim 3 wherein the PCR comprises an affinity moiety-labeled primer.

5. The method according to claim 4 wherein the identifying portions of the probes are incorporated in the resulting PCR amplicons, the method further comprising;
immobilizing affinity moiety-labeled amplicon strands with an affinity moiety binding partner;
removing reaction components lacking the affinity moiety;
hybridizing a plurality of mobility probes to the immobilized affinity moiety-labeled amplicon strands, wherein the mobility probes further comprise a region of complementary with the identifying portion or identifying portion complement of the affinity moiety-labeled amplicon strands;
removing unhybridized mobility probes;
eluting hybridized mobility probes; and,
analyzing the eluted mobility probes using a mobility dependent analysis technique, whereby the distinct mobility of the mobility probe determines the identity of the identifying portion.

6. The method according to claim 5 wherein the mobility probes further comprise distinguishable labels, wherein said distinguishable labels further comprise at least one florophore, wherein the florophore is at least one of 6FAM, dR6G, BigDye-Tamra, BigDye-Rox, and combinations thereof.

7. The method according to claim 5 wherein the mobility dependent analysis technique is capillary electrophoresis.

8. The method according to claim 4 wherein the affinity moiety is biotin.

9. The method according to claim 5 wherein the affinity moiety-binding partner is streptavidin.

10. The method according to claim 5 wherein a universal forward primer portion is incorporated into the first probes, wherein a universal reverse primer portion is incorporated into the second probes, wherein the PCR amplification comprises a set of universal primers that hybridize to their corresponding primer portions.

11. A method for detecting ligation in a ligation assay comprising,
providing a first reaction comprising a monomorphic target polynucleotide sequence and a positive control probe set, wherein the positive control probe set comprises a positive control first probe one, a positive control first probe two, and a second probe,
wherein the positive control first probe one comprises an identifying portion and a target specific portion complementary to the monomorphic polynucleotide sequence,
wherein the target specific portion comprises a discriminating region complementary to the corresponding nucleotide on the monomorphic polynucleotide sequence,
wherein the positive control first probe two comprises an identifying portion and a target specific portion complementary to the monomorphic polynucleotide sequence, wherein the target specific portion comprises a discriminating region complementary to the corresponding nucleotide on the monomorphic polynucleotide sequence,
wherein the identifying portion of the positive control first probe one differs from the identifying portion of the positive control first probe two,
wherein the second probe comprises a target specific portion;
hybridizing the monomorphic target polynucleotide sequence to the positive control first probe one, the positive control first probe two, and the positive control second probe, wherein the positive control first probes hybridize adjacent to the second probe;
ligating the positive control first probe one to the positive control second probe, ligating the positive control first probe two to the positive control second probe, thereby forming a positive control first probe one ligation product and a positive control first probe two ligation product;
detecting the positive control first probe one ligation product and the positive control first probe two ligation product based on their distinct identifying portions;
thereby assessing ligation.

12. The method according to claim 11 further comprising a second reaction, wherein the second reaction comprises a monomorphic target polynucleotide sequence and a negative control probe set, wherein the negative control probe set comprises a negative control first probe one, a negative control first probe two, and a second probe,
wherein the negative control first probe one comprises an identifying portion and a target specific portion complementary to the monomorphic polynucleotide sequence, wherein the target specific portion comprises a discriminating region that is not complementary to the corresponding nucleotide of the monomorphic target polynucleotide,
wherein the negative control first probe two comprises an identifying portion and a target specific portion complementary to the monomorphic polynucleotide sequence, wherein the target specific portion further comprises a discriminating region that is not complementary to the corresponding nucleotide of the monomorphic target polynucleotide,
wherein the identifying portion of the negative control first probe one differs from the identifying portion of the negative control first probe two,
wherein the second probe comprises a target specific portion,
hybridizing the monomorphic target polynucleotide sequence to the negative control first probe one, the negative control first probe two, and the negative control second probe, wherein the negative control first probes hybridize adjacent to the second probe;
ligating the negative control first probe one to the negative control second probe, ligating the negative control first probe two to the negative control second probe, thereby forming a negative control first probe one ligation product and a negative control first probe two ligation product;
detecting the negative control first probe one ligation product and the negative control first probe two ligation product based on their distinct identifying portions;
comparing the detected non-specific ligation product in the first reaction to the detected specific ligation products in the second reaction;
thereby assessing non-specific ligation in a ligation assay,

13. The method according to claim 12 wherein the ligation products are amplified by a PCR, wherein the PCR comprises a primer containing an affinity moiety, wherein the identifying portions of the probes and the affinity moiety of the primer are incorporated into a PCR amplicon, thereby forming an affinity-labeled PCR amplicon strand, the method further comprising;
immobilizing the affinity moiety-labeled amplicon strands with an affinity moiety binding partner;
removing reaction components lacking the affinity moiety;
hybridizing a mobility probe to the immobilized affinity moiety-labeled amplicon strands, wherein the mobility probe further comprises a region of complementary with the identifying portion or identifying portion complement of the affinity moiety-labeled amplicon strands;
removing unhybridized mobility probes;
eluting hybridized mobility probes; and,
analyzing the eluted mobility probes using a mobility dependent analysis technique, whereby the distinct mobility of the mobility probe determines the identity of the identifying portion, and hence an assessment of ligation.

14. The method according to claim 13 wherein the mobility probes further comprise distinguishable labels, wherein said distinguishable labels further comprise at least one florophore, wherein the florophore is at least one of 6FAM, dR6G, BigDye-Tamra, BigDye-Rox, and combinations thereof.

15. The method according to claim 13 wherein the mobility dependent analysis technique is capillary electrophoresis.

16. The method according to claim 13 wherein a universal forward primer portion is incorporated into the first probes, wherein a universal reverse primer portion is incorporated into the second probes, wherein the PCR amplification comprises a set of universal primers that hybridize to their corresponding primer portions.

17. The method according to claim 12 wherein the monomorphic target polynucleotide sequence queried in the first reaction with the positive control probe set is the same as the monomorphic target polynucleotide sequence queried in the second reaction with the negative control probe set.

18. The method according to claim 12 wherein the identifying portion of the positive control first probe one in the first positive control probe set is the same as the identifying portion of the negative control first probe one in the first negative control probe set, and wherein the identifying portion of the positive control first probe two in the first positive control probe set is the same as the identifying portion of the negative control first probe two in the first negative control probe set.

19. The method according to claim 18 wherein the monomorphic target polynucleotide sequence queried in the first reaction with the first positive control probe set is the same as the monomorphic target polynucleotide sequence queried in the second reaction with the negative control probe set, wherein the identifying portion of the positive control first probe one in the first positive control probe set is the same as the identifying portion of the negative control first probe one in the first negative control probe set, and wherein the identifying portion of the positive control first probe two in the first positive control probe set is the same as the identifying portion of the negative control first probe two in the first negative control probe set.

20. The method according to claim 19 wherein the first reaction and second reaction are performed in two different wells of the same microtitre plate.

21. A method for determining ligation specificity in a ligation assay comprising;
comparing the amount of a specific positive control ligation product to a non-specific negative control ligation product,
wherein the specific positive control ligation product results from ligating a first positive control probe to a second probe while hybridized on a monomorphic target polynucleotide,
wherein the non-specific negative control ligation product results from ligating a first negative control probe to a second probe while hybridized on a monomorphic target polynucleotide,
wherein the first positive control probe of the specific ligation product differs from the first negative control probe in the non-specific ligation product only by a discriminating region,
wherein the monomorphic target polynucleotide queried by the first positive control probe is the same as the monomorphic target polynucleotide queried by the first negative control probe;
quantifying the difference between the amount of the specific positive control ligation product and the non-specific negative control ligation product;
thereby determining ligation specificity in a ligation assay.

22. The method according to claim 21 comprising;
comparing the amount of an experimental ligation product to the amount of the specific positive control ligation product and the amount of the non-specific negative control ligation product,
wherein the experimental ligation product, the specific positive control ligation product, and the non-specific negative control ligation product are derived from the same ligation reaction
and determining therefrom the ligation specificity for the experimental ligation product in a ligation assay.

23. A method for assessing the variability of specific ligation in parallel ligation assays comprising;
comparing the amount of a specific positive control ligation product in a first reaction to a specific positive control ligation product in a second reaction,
wherein the specific positive control ligation product in the first reaction results from a positive control probe set, wherein the positive control probe set comprises a first positive control probe and a second probe,
wherein the specific positive control ligation product in the second reaction results from a positive control probe set, wherein the positive control probe set comprises a first positive control probe and a second probe,
wherein the first positive control probe of the first reaction does not differ from the first positive control probe in the second reaction, wherein the second probe of the first reaction does not differ from the second probe of the second reaction,
wherein a monomorphic target polynucleotide queried by the positive control probe set in the first reaction is the same as a monomorphic target polynucleotide queried by the positive control probe set in the second reaction;
quantifying the difference between the amount of the specific ligation product in the first reaction and the amount of the specific ligation product in the second reaction;
thereby assessing the variability of specific ligation in parallel ligation assays.

24. The method according to claim 23 wherein the first reaction comprises a plurality of positive control probe sets, wherein each positive control probe set in the first reaction queries a different monomorphic target polynucleotide, wherein the second reaction comprises a plurality of positive control probe sets, wherein each positive control probe set in the second reaction queries a different monomorphic target polynucleotide, wherein the monomorphic target polynucleotides queried in the first reaction are the same as the monomorphic target polynucleotides queried in the second reaction,
wherein the positive control first probe of the positive control probe set querying a given monomorphic target polynucleotide in the first reaction comprises an identifying portion,
wherein the positive control first probe of the positive control set querying a given monomorphic target polynucleotide in the second reaction comprises an identifying portion,
wherein the identifying portion of the positive control first probe in the first reaction querying a given monomorphic target polynucleotide is the same as the identifying portion of the positive control first probe in the second reaction querying that same monomorphic target polynucleotide.

25. The method according to claim 24 wherein each positive control probe set comprises a first positive control probe one and a first positive control probe two, wherein the first positive control probe one and the first positive control probe two each comprise an identifying portion, wherein the identifying portion of the first positive control probe one differs from the identifying portion of first positive probe two,
wherein the identifying protion of the positive control first probe one querying a given monomorphic target polynucleotide in the first reaction is the same as the identifying portion of the positive control first probe one querying that same monomorphic target polynucleotide in the second reaction,
wherein the identifying protion of the positive control first probe two querying a given monomorphic target polynucleotide in the first reaction is the same as the identifying portion of the positive control first probe two querying that same monomorphic target polynucleotide in the second reaction.

26. A method for assessing the variability of non-specific ligation in parallel ligation assays comprising;
comparing the amount of a non-specific negative control ligation product in a first reaction to a non-specific negative control ligation product in a second reaction;
wherein the non-specific negative control ligation product in the first reaction results from a negative control probe set, wherein the negative control probe set comprises a first negative control probe and a second probe,
wherein the non-specific negative control ligation product in the second reaction results from a negative control probe set, wherein the negative control probe set comprises a first negative control probe and a second probe,
wherein the first negative control probe of the first reaction does not differ from the first negative control probe in the second reaction, wherein the second probe of the first reaction does not differ from the second probe of the second reaction,
wherein a monomorphic target polynucleotide queried by the negative control probe set in the first reaction is the same as a monomorphic target polynucleotide queried by the negative control probe set in the second reaction;
quantifying the difference between the amount of the non-specific ligation product in the first reaction and the amount of non-specific ligation product in the second reaction;
thereby assessing the variability of non-specific ligation in a ligation assay.

27. The method according to claim 26 wherein the first reaction comprises a plurality of negative control probe sets, wherein each negative control probe set in the first reaction queries a different monomorphic target polynucleotide, wherein the second reaction comprises a plurality of negative control probe sets, wherein each negative control probe set in the second reaction queries a different monomorphic target polynucleotide, wherein the monomorphic target polynucleotides queried in the first reaction are the same as the monomorphic target polynucleotides queried in the second reaction,
wherein the negative control first probe of the negative control probe set querying a given monomorphic target polynucleotide in the first reaction comprises an identifying portion,
wherein the negative control first probe of the negative control set querying a given monomorphic target polynucleotide in the second reaction comprises an identifying portion,
wherein the identifying portion of the negative control first probe in the first reaction querying a given monomorphic target polynucleotide is the same as the identifying portion of the negative control first probe in the second reaction querying that same monomorphic target polynucleotide.

28. The method according to claim 27 wherein each negative control probe set comprises a first negative control probe one and a first negative control probe two, wherein the first negative control probe one and the first negative control probe two each comprise an identifying portion, wherein the identifying portion of first negative control probe one differs from the identifying portion of the first negative probe two,
wherein the identifying protion of the negative control first probe one querying a given monomorphic target polynucleotide in the first reaction is the same as the identifying portion of the negative control first probe one querying that same monomorphic target polynucleotide in the second reaction,
wherein the identifying protion of the negative control first probe two querying a given monomorphic target polynucleotide in the first reaction is the same as the identifying portion of the negative control first probe two querying that same monomorphic target polynucleotide in the second reaction.

29. A method for assessing the variability of non-specific and specific ligation in parallel ligation assays comprising;
comparing the amount of a non-specific negative control ligation product in a first reaction to a specific control ligation product in a second reaction;
wherein the non-specific negative control ligation product in the first reaction results from a negative control probe set, wherein the negative control probe set comprises a first negative control probe and a second probe,
wherein the specific positive control ligation product in the second reaction results from a positive control probe set, wherein the positive control probe set comprises a first positive control probe and a second probe,
wherein the first negative control probe of the first reaction differs from the first positive control probe in the second reaction by only a discriminating region, wherein the second probe of the first reaction does not differ from the second probe of the second reaction,
wherein a monomorphic target polynucleotide queried by the negative control probe set in the first reaction is the same as a monomorphic target polynucleotide queried by the positive control probe set in the second reaction;
quantifying the difference between the amount of the non-specific ligation product in the first reaction and the amount of specific ligation product in the second reaction;
thereby assessing the variability of specific and non-specific ligation in a ligation assay.

30. The method according to claim 29 wherein the first reaction comprises a plurality of negative control probe sets, wherein each negative control probe set in the first reaction queries a different monomorphic target polynucleotide, wherein the second reaction comprises a plurality of positive control probe sets, wherein each positive control probe set in the second reaction queries a different monomorphic target polynucleotide, wherein the monomorphic target polynucleotides queried in the first reaction are the same as the monomorphic target polynucleotides queried in the second reaction,
wherein the negative control first probe of the negative control probe set querying a given monomorphic target polynucleotide in the first reaction comprises an identifying portion,
wherein the negative control first probe of the positive control set querying a given monomorphic target polynucleotide in the second reaction comprises an identifying portion,
wherein the identifying portion of the negative control first probe in the first reaction querying a given monomorphic target polynucleotide is the same as the identifying portion of the positive control first probe in the second reaction querying that same monomorphic target polynucleotide.

31. The method according to claim 30 wherein each negative control probe set in the first reaction comprises a first negative control probe one and a first negative control probe two, wherein the first negative control probe one and the first negative control probe two each comprise an identifying portion, wherein the identifying portion of first negative control probe one differs from the identifying portion of the first negative probe two,
wherein each positive control probe set in the second reaction comprises a first positive control probe one and a first positive control probe two, wherein the first positive control probe one and the first positive control probe two each comprise an identifying portion, wherein the identifying portion of the positive control first probe one differs from the identifying portion of the first positive probe two,
wherein the identifying protion of the negative control first probe one querying a given monomorphic target polynucleotide in the first reaction is the same as the identifying portion of the positive control first probe one querying that same monomorphic target polynucleotide in the second reaction,
wherein the identifying protion of the negative control first probe two querying a given monomorphic target polynucleotide in the first reaction is the same as the identifying portion of the positive control first probe two querying that same monomorphic target polynucleotide in the second reaction.

32. A kit for assessing ligation comprising a positive control probe set, a negative control probe set, an experimental probe set, and combinations thereof.

33. A kit for assessing ligation comprising a plurality of positive control probe sets.

34. A kit of assessing ligation comprising a plurality of negative control probe sets.

35. A kit according to any of claims 32, 33, 34 further comprising a plurality of monomorphic target polynucleotides, a plurality of polymorphic target polynucleotides, a means for ligating, a means for phosphorylating, a means for amplifying, and combinations thereof.

36. A method of determining ligation specificity comprising the steps of,
hybridizing,
ligating,
amplifying,
removing,
separating,
detecting,
comparing,
and determining therefrom ligation specificity.
